(19) United States
(12) Patent Application Publication
(10) Pub. No.: US 2023/0381269 A1
(43) Pub. Date: Nov. 30, 2023

(54) METHODS OF TREATING EYE DISORDERS

(71) Applicant: Apellis Pharmaceuticals, Inc., Waltham, MA (US)

(72) Inventors: Ravikanth Metlapally, Wayland, MA (US); Ramiro Magalhaes Ribeiro, Arlington, MA (US)

(21) Appl. No.: 18/027,331

(22) PCT Filed: Sep. 21, 2021
(86) PCT No.: PCT/US21/51350

§ 371 Date: Mar. 20, 2023

Related U.S. Application Data
(60) Provisional application No. 63/145,392, filed on Feb. 3, 2021.
(60) Provisional application No. 63/081,277, filed on Sep. 21, 2020.

(51) Int. Cl. A61K 38/12; A61P 27/02; A61K 9/00; A61K 47/26

(52) U.S. Cl. CPC A61K 38/12; A61P 27/02; A61K 9/0048; A61K 9/0019; A61K 47/26

(57) ABSTRACT

Methods of treating eye disorders, e.g., age related macular degeneration, using C3 inhibitor, are disclosed.

### CROSS REFERENCE TO RELATED APPLICATIONS

This application claims the benefit of U.S. Provisional Application No. 63/081,277, filed Sep. 21, 2020, and U.S. Provisional Application No. 63/145,392, filed Feb. 3, 2021, the contents of all of which are hereby incorporated herein in their entirety.

### BACKGROUND

Complement is a system consisting of more than 30 plasma and cell-bound proteins that plays a significant role in both innate and adaptive immunity. The proteins of the complement system act in a series of enzymatic cascades through a variety of protein interactions and cleavage events. Complement activation occurs via three main pathways: the antibody-dependent classical pathway, the alternative pathway, and the mannose-binding lectin (MBL) pathway. Inappropriate or excessive complement activation is an underlying cause or contributing factor to a number of serious diseases and conditions, and considerable effort has been devoted over the past several decades to exploring various complement inhibitors as therapeutic agents.

### SUMMARY

In some aspects, the disclosure provides methods of treating a subject, comprising: detecting one or more drusen in at least one eye of the subject; and administering to the at least one eye of the subject an effective amount of a C3 inhibitor, wherein administering the C3 inhibitor reduces risk of the subject developing incomplete retinal pigment epithelium and outer retinal atrophy (iRORA) and/or complete retinal pigment epithelium and outer retinal atrophy (cRORA).

In some aspects, the disclosure provides methods of treating a subject, comprising: detecting incomplete retinal pigment epithelium and outer retinal atrophy (iRORA) in at least one eye of the subject; and administering to the at least one eye of the subject an effective amount of a C3 inhibitor, wherein administering the C3 inhibitor reduces risk of the subject developing complete retinal pigment epithelium and outer retinal atrophy (cRORA).

In some aspects, the disclosure provides methods of slowing or preventing development of iRORA and/or cRORA in a subject, the method comprising: detecting one or more drusen in at least one eye of the subject; and administering to the at least one eye of the subject an effective amount of a C3 inhibitor monthly or every other month for at least 6 months, 12 months, or more, wherein after administration of the C3 inhibitor for at least 6 months, 12 months, or more, the subject does not develop iRORA and/or cRORA.

In some aspects, the disclosure provides methods of slowing or preventing development of cRORA in a subject, the method comprising: detecting iRORA in at least one eye of the subject; and administering to the at least one eye of the subject an effective amount of a C3 inhibitor monthly or every other month for at least 6 months, 12 months, or more, wherein after administration of the C3 inhibitor for at least 6 months, 12 months, or more, the subject does not develop cRORA.

In some aspects, the disclosure provides methods of slowing or preventing development of iRORA and/or cRORA in a subject, the method comprising: detecting one or more drusen in at least one eye of the subject; and providing a treatment regimen to the subject, wherein the treatment regimen comprises administering to the at least one eye of the subject an effective amount of a C3 inhibitor monthly or every other month for at least 6 months, 12 months, or more, wherein the subject does not develop iRORA and/or cRORA for at least 6 months, 12 months, 18 months, 24 months, 30 months, 36 months, or more, after starting the treatment regimen.

In some aspects, the disclosure provides methods of slowing or preventing development of cRORA in a subject, the method comprising: detecting iRORA in at least one eye of the subject; and providing a treatment regimen to the subject, wherein the treatment regimen comprises administering to the at least one eye of the subject an effective amount of a C3 inhibitor monthly or every other month for at least 6 months, 12 months, or more, wherein the subject does not develop cRORA for at least 6 months, 12 months, 18 months, 24 months, 30 months, 36 months, or more, after starting the treatment regimen.

In some aspects, the disclosure provides methods of slowing or preventing development of iRORA and/or cRORA in a subject for at least 6 months, 12 months, 18 months, 24 months, 30 months, 36 months, or more, after providing a treatment regimen to the subject, the method comprising: detecting one or more drusen in at least one eye of the subject; and administering the treatment regimen to the subject, wherein the treatment regimen comprises administering to the at least one eye of the subject an effective amount of a C3 inhibitor monthly or every other month for at least 6 months, 12 months, or more, thereby slowing or preventing development of iRORA and/or cRORA for at least 6 months, 12 months, 18 months, 24 months, 30 months, 36 months, or more, after starting the treatment regimen.

In some aspects, the disclosure provides methods of slowing or preventing development of cRORA in a subject for at least 6 months, 12 months, 18 months, 24 months, 30 months, 36 months, or more, after providing a treatment regimen to the subject, the method comprising: detecting iRORA in at least one eye of the subject; and administering the treatment regimen to the subject, wherein the treatment regimen comprises administering to the at least one eye of the subject an effective amount of a C3 inhibitor monthly or every other month for at least 6 months, 12 months, or more, thereby slowing or preventing development of cRORA for at least 6 months, 12 months, 18 months, 24 months, 30 months, 36 months, or more, after starting the treatment regimen.

In any of the aspects disclosed herein, the one or more drusen are greater than 20 (e.g., greater than 30, 35, 40, 45, 50, 55, 60, 70, or 80) microns in size. In some embodiments, the one or more drusen are at least 40 microns in height as determined by OCT.

In any of the aspects disclosed herein, OCT is used to assess iRORA, and presence of iRORA is determined by: (i) detecting a region of signal hypertransmission into the choroid of less than about 250 μm in diameter; (ii) detecting a corresponding zone of attenuation or disruption of the retinal pigment epithelium (RPE) of less than about 250 μm in diameter (e.g., with or without persistence of basal laminar deposits); and (iii) detecting evidence of overlying photoreceptor degeneration (e.g., subsidence of the inner nuclear layer (INL) and outer plexiform layer (OPL), presence of a hyporeflective wedge in the Henle fiber layer (HFL), thinning of the outer nuclear layer (ONL), disruption of the external limiting membrane (ELM), and/or disintegrity of the ellipsoid zone (EZ)).

In any of the aspects disclosed herein, OCT is used to assess cRORA, and presence of cRORA is determined by: (i) detecting a region of hypertransmission of at least 250 μm in diameter; (ii) detecting a zone of attenuation or disruption of the RPE of at least 250 μm in diameter; and (iii) detecting evidence of overlying photoreceptor degeneration (e.g., subsidence of the INL and OPL, presence of a hyporeflective wedge in the HFL, thinning of the ONL, disruption of the ELM, and/or disintegrity of the EZ.

In any of the aspects disclosed herein, the subject has or is suffering from early age-related macular degeneration (AMD) or intermediate AMD.

In any of the aspects disclosed herein, the C3 inhibitor is a compstatin analog described herein, e.g., pegcetacoplan.

In any of the aspects disclosed herein, the C3 inhibitor (e.g., compstatin analog, e.g., pegcetacoplan) is administered (e.g., at a dose of about 10 mg to about 20 mg, e.g., about 15 mg) as a composition comprising trehalose.

### BRIEF DESCRIPTION OF THE DRAWING

FIG. 1 shows the structure of pegcetacoplan (“APL-2”), assuming n of about 800 to about 1100 and a PEG of about 40 kD.

FIG. 2 depicts the LS mean (±SE) change from baseline in square root GA lesion (mm) in subjects receiving APL-2 monthly, subjects receiving APL-2 every other month, or sham pooled subjects. *Square root. Modified intention-to-treat (mITT) population was used for the efficacy analysis; defined as all patients who received at least 1 injection and underwent at least 1 follow-up examination at month 2 or later at which primary efficacy data were collected. 2-sided t tests at the alpha=0.1 level.

FIG. 3 depicts adverse event profile for GA subjects receiving APL-2 monthly, subjects receiving APL-2 every other month, or sham pooled subjects.

FIG. 4 depicts baseline characteristics from post-hoc analysis of GA subjects receiving APL-2 monthly, APL-2 every other month, or sham pooled subjects.

FIG. 5A depicts progression from large druse to iRORA or cRORA from post-hoc analysis of GA subjects receiving APL-2 monthly or sham pooled subjects. Pearson Chi-Square: Month 6—P=0.65; Month 12—P=0.31.

FIG. 5B depicts progression from iRORA to cRORA from post-hoc analysis of GA subjects receiving APL-2 monthly, APL-2 every other month, or sham pooled subjects. Pearson Chi-Square: Month 6—P=0.08 for PM; P=0.32 for PEOM; Month 12—P=0.02 for PM; P=0.03 for PEOM. Relative risk: Month 6—0.52 (0.23-1.18) for PM; 0.76 (0.45-1.29) for PEOM; Month 12—0.61 (0.37-1.00) for PM; 0.70 (0.50-0.98) for PEOM.

FIG. 5C depicts progression from iRORA to cRORA from post-hoc analysis (lesion level data) of GA subjects receiving APL-2 monthly, APL-2 every other month, or sham pooled subjects. Relative risk: Month 6—0.53 (0.24-1.14) for PM; 0.51 (0.30-0.87) for PEOM; Month 12—0.59 (0.36-0.97) for PM; 0.60 (0.43-0.82) for PEOM.

### DETAILED DESCRIPTION OF CERTAIN EMBODIMENTS

### Definitions

Antibody: As used herein, the term “antibody” refers to an immunoglobulin or a derivative thereof containing an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the invention the antibody is a fragment such as an Fab′, F(ab′)2, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. The antibody may be partially or completely humanized. An antibody may be polyclonal or monoclonal, though for purposes of the present invention monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Subject: As used herein, the term “subject” refers to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In some embodiments a subject suffering from a disease, e.g., AMD, may be referred to as a “patient”.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition. A subject suffering from a disease, disorder, and/or condition may be said to be in need of treatment for the disease, disorder, and/or condition, and vice versa.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed AMD or is at increased risk of developing such a disorder relative to a member of the general population. A composition of the disclosure can be administered prophylactically, i.e., before development of any symptom or manifestation of the condition. Typically in this case the subject will be at risk of developing the condition.

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, the prefix poly—refers to a nucleic acid containing 2 to about 10,000, 2 to about 50,000, or 2 to about 100,000 nucleotide monomer units. In some embodiments, the prefix oligo—refers to a nucleic acid containing 2 to about 200 nucleotide monomer units.

### II. Complement System

Complement is a system consisting of numerous plasma and cell-bound proteins that plays a significant role in both innate and adaptive immunity. The proteins of the complement system act in a series of enzymatic cascades through a variety of protein interactions and cleavage events. To facilitate understanding of the disclosure, and without intending to limit the invention in any way, this section provides an overview of complement and its pathways of activation. Further details are found, e.g., in Kuby Immunology, 6^{th} ed., 2006; Paul, W. E., Fundamental Immunology, Lippincott Williams & Wilkins; 6th ed., 2008; and Walport M J., Complement. First of two parts. N Engl J Med., 344(14):1058-66, 2001.

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. It will be understood that the names “C2a” and “C2b” used initially were subsequently reversed in the scientific literature.

The alternative pathway is initiated by and amplified at, e.g., microbial surfaces and various complex polysaccharides. In this pathway, hydrolysis of C3 to C3(H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by host cell molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on host cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death. If the TCC does not insert into a membrane, it can circulate in the blood as soluble sC5b-9 (sC5b-9). Levels of sC5b-9 in the blood may serve as an indicator of complement activation.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Inappropriate or excessive complement activation is an underlying cause or contributing factor to a number of serious diseases and conditions. Deficiencies or defects in complement regulatory protein(s) are involved in the pathogenesis of a variety of complement-mediated disorders.

### III. Compstatin Analogs

In some embodiments, a C3 inhibitor administered to treat an eye disorder described herein comprises a compstatin analog. Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus. The term “compstatin” is used herein consistently with such usage. Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), WO/2009/046198 (PCT/US2008/078593); WO/2010/127336 (PCT/US2010/033345).

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

Compounds “designed or identified based on compstatin” include, but are not limited to, compounds that comprise an amino acid chain whose sequence is obtained by (i) modifying the sequence of compstatin (e.g., replacing one or more amino acids of the sequence of compstatin with a different amino acid or amino acid analog, inserting one or more amino acids or amino acid analogs into the sequence of compstatin, or deleting one or more amino acids from the sequence of compstatin); (ii) selection from a phage display peptide library in which one or more amino acids of compstatin is randomized, and optionally further modified according to method (i); or (iii) identified by screening for compounds that compete with compstatin or any analog thereof obtained by methods (i) or (ii) for binding to C3 or a fragment thereof. Many useful compstatin analogs comprise a hydrophobic cluster, a β-turn, and a disulfide bridge.

In certain embodiments, sequence of a compstatin analog comprises or consists essentially of a sequence that is obtained by making 1, 2, 3, or 4 substitutions in the sequence of compstatin, i.e., 1, 2, 3, or 4 amino acids in the sequence of compstatin is replaced by a different standard amino acid or by a non-standard amino acid. In certain embodiments, the amino acid at position 4 is altered. In certain embodiments, the amino acid at position 9 is altered. In certain embodiments, the amino acids at positions 4 and 9 are altered. In certain embodiments, only the amino acids at positions 4 and 9 are altered. In certain embodiments, the amino acid at position 4 or 9 is altered, or in certain embodiments both amino acids 4 and 9 are altered, and in addition up to 2 amino acids located at positions selected from 1, 7, 10, 11, and 13 are altered. In certain embodiments, the amino acids at positions 4, 7, and 9 are altered. In certain embodiments, amino acids at position 2, 12, or both are altered, provided that the alteration preserves the ability of the compound to be cyclized. Such alteration(s) at positions 2 and/or 12 may be in addition to the alteration(s) at position 1, 4, 7, 9, 10, 11, and/or 13. Optionally the sequence of any of the compstatin analogs whose sequence is obtained by replacing one or more amino acids of compstatin sequence further includes up to 1, 2, or 3 additional amino acids at the C-terminus. In one embodiment, the additional amino acid is Gly. Optionally the sequence of any of the compstatin analogs whose sequence is obtained by replacing one or more amino acids of compstatin sequence further includes up to 5, or up to 10 additional amino acids at the C-terminus. It should be understood that compstatin analogs may have any one or more of the characteristics or features of the various embodiments described herein, and characteristics or features of any embodiment may additionally characterize any other embodiment described herein, unless otherwise stated or evident from the context. In certain embodiments, the sequence of a compstatin analog comprises or consists essentially of a sequence identical to that of compstatin except at positions corresponding to positions 4 and 9 in the sequence of compstatin.

Compstatin and certain compstatin analogs having somewhat greater activity than compstatin contain only standard amino acids (“standard amino acids” are glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine and histidine). Certain compstatin analogs having improved activity incorporate one or more non-standard amino acids. Useful non-standard amino acids include singly and multiply halogenated (e.g., fluorinated) amino acids, D-amino acids, homo-amino acids, N-alkyl amino acids, dehydroamino acids, aromatic amino acids (other than phenylalanine, tyrosine and tryptophan), ortho-, meta- or para-aminobenzoic acid, phospho-amino acids, methoxylated amino acids, and α,α-disubstituted amino acids. In certain embodiments, a compstatin analog is designed by replacing one or more L-amino acids in a compstatin analog described elsewhere herein with the corresponding D-amino acid. Such compounds and methods of use thereof are an aspect of the disclosure. Exemplary non-standard amino acids of use include 2-naphthylalanine (2-NaI), 1-naphthylalanine (1-NaI), 2-indanylglycine carboxylic acid (2Ig1), dihydrotrpytophan (Dht), 4-benzoyl-L-phenylalanine (Bpa), 2-α-aminobutyric acid (2-Abu), 3-α-aminobutyric acid (3-Abu), 4-α-aminobutyric acid (4-Abu), cyclohexylalanine (Cha), homocyclohexylalanine (hCha), 4-fluoro-L-tryptophan (4fW), 5-fluoro-L-tryptophan (5fW), 6-fluoro-L-tryptophan (6fW), 4-hydroxy-L-tryptophan (4OH—W), 5-hydroxy-L-tryptophan (5OH—W), 6-hydroxy-L-tryptophan (6OH—W), 1-methyl-L-tryptophan (1MeW), 4-methyl-L-tryptophan (4MeW), 5-methyl-L-tryptophan (5MeW), 7-aza-L-tryptophan (7aW), α-methyl-L-tryptophan (αMeW), β-methyl-L-tryptophan (βMeW), N-methyl-L-tryptophan (NMeW), ornithine (orn), citrulline, norleucine, γ-glutamic acid, etc.

In certain embodiments, the compstatin analog comprises one or more Trp analogs (e.g., at position 4 and/or 7 relative to the sequence of compstatin). Exemplary Trp analogs are mentioned above. See also Beene, et. al. Biochemistry 41: 10262-10269, 2002 (describing, inter alia, singly- and multiply-halogenated Trp analogs); Babitzke & Yanofsky, J. Biol. Chem. 270: 12452-12456, 1995 (describing, inter alia, methylated and halogenated Trp and other Trp and indole analogs); and U.S. Pat. Nos. 6,214,790, 6,169,057, 5,776,970, 4,870,097, 4,576,750 and 4,299,838. Other Trp analogs include variants that are substituted (e.g., by a methyl group) at the α or β carbon and, optionally, also at one or more positions of the indole ring. Amino acids comprising two or more aromatic rings, including substituted, unsubstituted, or alternatively substituted variants thereof, are of interest as Trp analogs. In certain embodiments, the Trp analog, e.g., at position 4, is 5-methoxy, 5-methyl-, 1-methyl-, or 1-formyl-tryptophan. In certain embodiments, a Trp analog (e.g., at position 4) comprising a 1-alkyl substituent, e.g., a lower alkyl (e.g., C1-C5) substituent is used. In certain embodiments, N(α) methyl tryptophan or 5-methyltryptophan is used. In some embodiments, an analog comprising a 1-alkanyol substituent, e.g., a lower alkanoyl (e.g., C₁-C₅) is used. Examples include 1-acetyl-L-tryptophan and L-β-tryptophan.

In certain embodiments, the Trp analog has increased hydrophobic character relative to Trp. For example, the indole ring may be substituted by one or more alkyl (e.g., methyl) groups. In certain embodiments, the Trp analog participates in a hydrophobic interaction with C3. Such a Trp analog may be located, e.g., at position 4 relative to the sequence of compstatin. In certain embodiments, the Trp analog comprises a substituted or unsubstituted bicyclic aromatic ring component or two or more substituted or unsubstituted monocyclic aromatic ring components.

In certain embodiments, the Trp analog has increased propensity to form hydrogen bonds with C3 relative to Trp but does not have increased hydrophobic character relative to Trp. The Trp analog may have increased polarity relative to Trp and/or an increased ability to participate in an electrostatic interaction with a hydrogen bond donor on C3. Certain exemplary Trp analogs with an increased hydrogen bond forming character comprise an electronegative substituent on the indole ring. Such a Trp analog may be located, e.g., at position 7 relative to the sequence of compstatin.

In certain embodiments, the compstatin analog comprises one or more Ala analogs (e.g., at position 9 relative to the sequence of compstatin), e.g., Ala analogs that are identical to Ala except that they include one or more CH₂ groups in the side chain. In certain embodiments, the Ala analog is an unbranched single methyl amino acid such as 2-Abu. In certain embodiments, the compstatin analog comprises one or more Trp analogs (e.g., at position 4 and/or 7 relative to the sequence of compstatin) and an Ala analog (e.g., at position 9 relative to the sequence of compstatin).

In certain embodiments, the compstatin analog is a compound that comprises a peptide that has a sequence of (X′aa)ₙ-Gln-Asp-Xaa-Gly-(X″aa)ₘ, (SEQ ID NO: 2) wherein each X′aa and each X″aa is an independently selected amino acid or amino acid analog, wherein Xaa is Trp or an analog of Trp, and wherein n>1 and m>1 and n+m is between 5 and 21. The peptide has a core sequence of Gln-Asp-Xaa-Gly, where Xaa is Trp or an analog of Trp, e.g., an analog of Trp having increased propensity to form hydrogen bonds with an H-bond donor relative to Trp but, in certain embodiments, not having increased hydrophobic character relative to Trp. For example, the analog may be one in which the indole ring of Trp is substituted with an electronegative moiety, e.g., a halogen such as fluorine. In one embodiment, Xaa is 5-fluorotryptophan. Absent evidence to the contrary, one of skill in the art would recognize that any non-naturally occurring peptide whose sequence comprises this core sequence and that inhibits complement activation and/or binds to C3 will have been designed based on the sequence of compstatin. In an alternative embodiment, Xaa is an amino acid or amino acid analog other than a Trp analog that allows the Gln-Asp-Xaa-Gly peptide to form a β-turn.

In certain embodiments, the peptide has a core sequence of X′aa-Gln-Asp-Xaa-Gly (SEQ ID NO: 3), where X′aa and Xaa are selected from Trp and analogs of Trp. In certain embodiments, the peptide has a core sequence of X′aa-Gln-Asp-Xaa-Gly (SEQ ID NO: 3), where X′aa and Xaa are selected from Trp, analogs of Trp, and other amino acids or amino acid analogs comprising at least one aromatic ring. In certain embodiments, the core sequence forms a β-turn in the context of the peptide. The β—turn may be flexible, allowing the peptide to assume two or more conformations as assessed for example, using nuclear magnetic resonance (NMR). In certain embodiments, X′aa is an analog of Trp that comprises a substituted or unsubstituted bicyclic aromatic ring component or two or more substituted or unsubstituted monocyclic aromatic ring components. In certain embodiments, X′aa is selected from the group consisting of 2-napthylalanine, 1-napthylalanine, 2-indanylglycine carboxylic acid, dihydrotryptophan, and benzoylphenylalanine. In certain embodiments, X′aa is an analog of Trp that has increased hydrophobic character relative to Trp. For example, X′aa may be 1-methyltryptophan. In certain embodiments, Xaa is an analog of Trp that has increased propensity to form hydrogen bonds relative to Trp but, in certain embodiments, not having increased hydrophobic character relative to Trp. In certain embodiments, the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan.

In certain embodiments, the peptide has a core sequence of X′aa-Gln-Asp-Xaa-Gly-X″aa (SEQ ID NO: 4), where X′aa and Xaa are each independently selected from Trp and analogs of Trp and X″aa is selected from His, Ala, analogs of Ala, Phe, and Trp. In certain embodiments, X′aa is an analog of Trp that has increased hydrophobic character relative to Trp, such as 1-methyltryptophan or another Trp analog having an alkyl substituent on the indole ring (e.g., at position 1, 4, 5, or 6). In certain embodiments, X′aa is an analog of Trp that comprises a substituted or unsubstituted bicyclic aromatic ring component or two or more substituted or unsubstituted monocyclic aromatic ring components. In certain embodiments, X′aa is selected from the group consisting of 2-napthylalanine, 1-napthylalanine, 2-indanylglycine carboxylic acid, dihydrotryptophan, and benzoylphenylalanine. In certain embodiments, Xaa is an analog of Trp that has increased propensity to form hydrogen bonds with C3 relative to Trp but, in certain embodiments, not having increased hydrophobic character relative to Trp. In certain embodiments, the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan. In certain embodiments, X″aa is Ala or an analog of Ala such as Abu or another unbranched single methyl amino acid. In certain embodiments, the peptide has a core sequence of X′aa-Gln-Asp-Xaa-Gly-X″aa (SEQ ID NO: 4), where X′aa and Xaa are each independently selected from Trp, analogs of Trp, and amino acids or amino acid analogs comprising at least one aromatic side chain, and X″aa is selected from His, Ala, analogs of Ala, Phe, and Trp. In certain embodiments, X″aa is selected from analogs of Trp, aromatic amino acids, and aromatic amino acid analogs.

In certain preferred embodiments, the peptide is cyclic. The peptide may be cyclized via a bond between any two amino acids, one of which is (X′aa)ₙ and the other of which is located within (X″aa)ₘ. In certain embodiments, the cyclic portion of the peptide is between 9 and 15 amino acids in length, e.g., 10-12 amino acids in length. In certain embodiments, the cyclic portion of the peptide is 11 amino acids in length, with a bond (e.g., a disulfide bond) between amino acids at positions 2 and 12. For example, the peptide may be 13 amino acids long, with a bond between amino acids at positions 2 and 12 resulting in a cyclic portion 11 amino acids in length.

In certain embodiments, the peptide comprises or consists of the sequence X′aa1-X′aa2-X′aa3-X′aa4-Gln-Asp-Xaa-Gly-X″aa1-X″aa2-X″aa3-X″aa4-X″aa5 (SEQ ID NO: 5). In certain embodiments, X′aa4 and Xaa are selected from Trp and analogs of Trp, and X′aa1, X′aa2, X′aa3, X″aa1, X″aa2, X″aa3, X″aa4, and X″aa5 are independently selected from among amino acids and amino acid analogs. In certain embodiments, X′aa4 and Xaa are selected from aromatic amino acids and aromatic amino acid analogs. Any one or more of X′aa1, X′aa2, X′aa3, X″aa1, X″aa2, X″aa3, X″aa4, and X″aa5 may be identical to the amino acid at the corresponding position in compstatin. In one embodiment, X″aa1 is Ala or a single methyl unbranched amino acid. The peptide may be cyclized via a covalent bond between (i) X′aa1, X′aa2, or X′aa3; and (ii) X″aa2, X″aa3, X″aa4 or X″aa5. In one embodiment the peptide is cyclized via a covalent bond between X′aa2 and X″aa4. In one embodiment, the covalently bound amino acid are each Cys and the covalent bond is a disulfide (S—S) bond. In other embodiments, the covalent bond is a C—C, C—O, C—S, or C—N bond. In certain embodiments, one of the covalently bound residues is an amino acid or amino acid analog having a side chain that comprises a primary or secondary amine, the other covalently bound residue is an amino acid or amino acid analog having a side chain that comprises a carboxylic acid group, and the covalent bond is an amide bond. Amino acids or amino acid analogs having a side chain that comprises a primary or secondary amine include lysine and diaminocarboxylic acids of general structure NH₂(CH₂)ₙCH(NH₂)COOH such as 2,3-diaminopropionic acid (dapa), 2,4-diaminobutyric acid (daba), and ornithine (orn), wherein n=1 (dapa), 2 (daba), and 3 (orn), respectively. Examples of amino acids having a side chain that comprises a carboxylic acid group include dicarboxylic amino acids such as glutamic acid and aspartic acid. Analogs such as beta-hydroxy-L-glutamic acid may also be used. In some embodiments, a peptide is cyclized with a thioether bond, e.g., as described in PCT/US2011/052442 (WO/2012/040259). For example, in some embodiments a disulfide bond in any of the peptides is replaced with a thioether bond. In some embodiments, a cystathionine is formed. In some embodiments, the cystathionine is a delta-cystathionine or a gamma-cystathionine. In some embodiments, a modification comprises replacement of a Cys-Cys disulfide bond between cysteines at X′aa2 and X″aa4 in SEQ ID NO: 5 (or corresponding positions in other sequences) with addition of a CH₂, to form a homocysteine at X′aa2 or X″aa4, and introduction of a thioether bond, to form a cystathionine. In one embodiment, the cystathionine is a gamma-cystathionine. In another embodiment, the cystathionine is a delta-cystathionine. Another modification in accordance with the present disclosure comprises replacement of the disulfide bond with a thioether bond without the addition of a CH₂, thereby forming a lantithionine. In some embodiments, a compstatin analog having a thioether in place of a disulfide bond has increased stability, at least under some conditions, as compared with the compstatin analog having the disulfide bond.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 6); wherein:
Xaa1 is Ile, Val, Leu, B¹-Val, B¹-Leu or a dipeptide comprising Gly-Ile or B¹-Gly-Ile, and β¹ represents a first blocking moiety;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of the L-Thr, D-Thr, Ile, Val, Gly, Ala, or Asn optionally is replaced by a second blocking moiety B²; and the two Cys residues are joined by a disulfide bond. In some embodiments, Xaa4 is Leu, Nle, His, or Phe or a dipeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B²; and the two Cys residues are joined by a disulfide bond.

In other embodiments, Xaa1 is absent or is any amino acid or amino acid analog, and Xaa2, Xaa2*, Xaa3, and Xaa4 are as defined above. If Xaa1 is absent, the N-terminal Cys residue may have a blocking moiety B¹ attached thereto.

In another embodiment, Xaa4 is any amino acid or amino acid analog and Xaa1, Xaa2, Xaa2*, and Xaa3 are as defined above. In another embodiment Xaa4 is a dipeptide selected from the group consisting of: Thr-Ala and Thr-Asn, wherein the carboxy terminal —OH or the Ala or Asn is optionally replaced by a second blocking moiety B².

In any of the embodiments of the compstatin analog of SEQ ID NO: 6, Xaa2 may be Trp.

In any of the embodiments of the compstatin analog of SEQ ID NO: 6, Xaa2 may be an analog of Trp comprising a substituted or unsubstituted bicyclic aromatic ring component or two or more substituted or unsubstituted monocyclic aromatic ring components. For example, the analog of Trp may be selected from 2-naphthylalanine (2-NaI), 1-naphthylalanine (1-NaI), 2-indanylglycine carboxylic acid (Ig1), dihydrotrpytophan (Dht), and 4-benzoyl-L-phenylalanine.

In any of the embodiments of the compstatin analog of SEQ ID NO: 6, Xaa2 may be an analog of Trp having increased hydrophobic character relative to Trp. For example, the analog of Trp may be selected from 1-methyltryptophan, 4-methyltryptophan, 5-methyltryptophan, and 6-methyltryptophan. In one embodiment, the analog of Trp is 1-methyltryptophan. In one embodiment, Xaa2 is 1-methyltryptophan, Xaa2* is Trp, Xaa3 is Ala, and the other amino acids are identical to those of compstatin.

In any of the embodiments of the compstatin analog of SEQ ID NO: 6, Xaa2* may be an analog of Trp such as an analog of Trp having increased hydrogen bond forming propensity with C3 relative to Trp, which, in certain embodiments, does not have increased hydrophobic character relative to Trp. In certain embodiments the analog of Trp comprises an electronegative substituent on the indole ring. For example, the analog of Trp may be selected from 5-fluorotryptophan and 6-fluorotryptophan.

In certain embodiments Xaa2 is Trp and Xaa2* is an analog of Trp having increased hydrogen bond forming propensity with C3 relative to Trp which, in certain embodiments, does not have increased hydrophobic character relative to Trp. In certain embodiments of the compstatin analog of SEQ ID NO: 6, Xaa2 is analog of Trp having increased hydrophobic character relative to Trp such as an analog of Trp selected from 1-methyltryptophan, 4-methyltryptophan, 5-methyltryptophan, and 6-methyltryptophan, and Xaa2* is an analog of Trp having increased hydrogen bond forming propensity with C3 relative to Trp which, in certain embodiments, does not have increased hydrophobic character relative to Trp. For example, in one embodiment Xaa2 is methyltryptophan and Xaa2* is 5-fluorotryptophan.

In certain of the afore-mentioned embodiments, Xaa3 is Ala. In certain of the afore-mentioned embodiments Xaa3 is a single methyl unbranched amino acid, e.g., Abu.

The disclosure further provides compstatin analogs of SEQ ID NO: 6, as described above, wherein Xaa2 and Xaa2* are independently selected from Trp, analogs of Trp, and other amino acids or amino acid analogs that comprise at least one aromatic ring, and Xaa3 is His, Ala or an analog of Ala, Phe, Trp, an analog of Trp, or another aromatic amino acid or aromatic amino acid analog.

In certain embodiments, the blocking moiety present at the N- or C-terminus of any of the compstatin analogs described herein is any moiety that stabilizes a peptide against degradation that would otherwise occur in mammalian (e.g., human or non-human primate) blood or interstitial fluid. For example, blocking moiety B¹ could be any moiety that alters the structure of the N-terminus of a peptide so as to inhibit cleavage of a peptide bond between the N-terminal amino acid of the peptide and the adjacent amino acid. Blocking moiety B² could be any moiety that alters the structure of the C-terminus of a peptide so as to inhibit cleavage of a peptide bond between the C-terminal amino acid of the peptide and the adjacent amino acid. Any suitable blocking moieties known in the art could be used. In certain embodiments of the invention blocking moiety B¹ comprises an acyl group (i.e., the portion of a carboxylic acid that remains following removal of the —OH group). The acyl group typically comprises between 1 and 12 carbons, e.g., between 1 and 6 carbons. For example, in certain embodiments of the invention blocking moiety B¹ is selected from the group consisting of: formyl, acetyl, proprionyl, butyryl, isobutyryl, valeryl, isovaleryl, etc. In one embodiment, the blocking moiety B¹ is an acetyl group, i.e., Xaa1 is Ac-Ile, Ac-Val, Ac-Leu, or Ac-Gly-Ile.

In certain embodiments blocking moiety B² is a primary or secondary amine (—NH₂ or —NHR¹, wherein R is an organic moiety such as an alkyl group).

In certain embodiments blocking moiety B¹ is any moiety that neutralizes or reduces the positive charge that may otherwise be present at the N-terminus at physiological pH. In certain embodiments of the invention blocking moiety B² is any moiety that neutralizes or reduces the negative charge that may otherwise be present at the C-terminus at physiological pH.

In certain embodiments the compstatin analog is acetylated or amidated at the N-terminus and/or C-terminus, respectively. A compstatin analog may be acetylated at the N-terminus, amidated at the C-terminus, and or both acetylated at the N-terminus and amidated at the C-terminus. In certain embodiments a compstatin analog comprises an alkyl or aryl group at the N-terminus rather than an acetyl group.

In certain embodiments, the compstatin analog is a compound that comprises a peptide having a sequence:

(SEQ ID NO: 7)
Xaa1-Cys-Val-Xaa2-Gln-Asp-Xaa2*-Gly-Xaa3-His-Arg-
Cys-Xaa4;
wherein:
Xaa1 is Ile, Val, Leu, Ac-Ile, Ac-Val, Ac-Leu
or a dipeptide comprising Gly-Ile or Ac-Gly-Ile;

Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of L-Thr, D-Thr, Ile, Val, Gly, Ala, or Asn optionally is replaced by —NH₂; and the two Cys residues are joined by a disulfide bond. In some embodiments, Xaa4 is Leu, Nle, His, or Phe or a dipeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B²; and the two Cys residues are joined by a disulfide bond.

In some embodiments, Xaa1, Xaa2, Xaa2*, Xaa3, and Xaa4 are as described above for the various embodiments of SEQ ID NO: 6. For example, in certain embodiments Xaa2* is Trp. In certain embodiments, Xaa2 is an analog of Trp having increased hydrophobic character relative to Trp, e.g., 1-methyltryptophan. In certain embodiments Xaa3 is Ala. In certain embodiments Xaa3 is a single methyl unbranched amino acid.

In certain embodiments, Xaa1 is Ile and Xaa4 is L-Thr.

In certain embodiments, Xaa1 is Ile, Xaa2* is Trp, and Xaa4 is L-Thr.

The disclosure further provides compstatin analogs of SEQ ID NO: 7, as described above, wherein Xaa2 and Xaa2* are independently selected from Trp, analogs of Trp, other amino acids or aromatic amino acid analogs, and Xaa3 is His, Ala or an analog of Ala, Phe, Trp, an analog of Trp, or another aromatic amino acid or aromatic amino acid analog.

In certain embodiments of any of the compstatin analogs described herein, an analog of Phe is used rather than Phe.

Table 1 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus. Bold text is used to indicate certain modifications. Activity relative to compstatin is based on published data and assays described therein (WO2004/026328, WO2007044668, Mallik, 2005; Katragadda, 2006). In certain embodiments, the peptides listed in Table 1 are cyclized via a disulfide bond between the two Cys residues when used in the therapeutic compositions and methods of the disclosure. Alternate means for cyclizing the peptides are also within the scope of the disclosure. in various embodiments of the invention one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments of the invention, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in Table 1 contains at least one N-methyl isoleucine, e.g., at the position corresponding to position 13 of compstatin. For example, a Thr at or near the C-terminal end of a peptide whose sequence is listed in Table 1 or any other compstatin analog sequence may be replaced by N-methyl Ile. As will be appreciated, in some embodiments the N-methylated amino acids comprise N-methyl Gly at position 8 and N-methyl Ile at position 13. In some embodiments the N-methylated amino acids comprise N-methyl Gly in a core sequence such as SEQ ID NO: 3 or SEQ ID NO: 4. In some embodiments the N-methylated amino acids comprise N-methyl Gly in a core sequence such as SEQ ID NO: 5, SEQ ID NO: 6, or SEQ ID NO: 7.

TABLE 1

| | | SEQ | Activity |
|---|---|---|---|
| | | ID | over |
| Peptide | Sequence | NO: | compstatin |
| Compstatin | H-ICVVQDWGHHRCT-CONH2 | 8 | * |
| Ac-compstatin | Ac-ICVVQDWGHHRCT-CONH2 | 9 | 3xmore |
| Ac-V4Y/H9A | Ac-ICVYQDWGAHRCT-CONH2 | 10 | 14xmore |
| Ac-V4W/H9A-OH | Ac-ICVWQDWGAHRCT-COOH | 11 | 27xmore |
| Ac-V4W/H9A | Ac-ICVWQDWGAHRCT-CONH2 | 12 | 45xmore |
| Ac-V4W/H9A/T13dT-OH | Ac-ICVWQDWGAHRCdT-COOH | 13 | 55xmore |
| Ac-V4(2-Nal)/H9A | Ac-ICV(2-Nal)QDWGAHRCT-CONH2 | 14 | 99xmore |
| Ac V4(2-Nal)/H9A-OH | Ac-ICV(2-Nal)QDWGAHRCT-COOH | 15 | 38xmore |
| Ac V4(1-Nal)/H9A-OH | Ac-ICV(1-Nal)QDWGAHRCT-COOH | 16 | 30xmore |
| Ac-V42Igl/H9A | Ac-ICV(2-Igl)QDWGAHRCT-CONH2 | 17 | 39xmore |
| Ac-V42Igl/H9A-OH | Ac-ICV(2-Igl)QDWGAHRCT-COOH | 18 | 37xmore |
| Ac-V4Dht/H9A-OH | Ac-ICVDhtQDWGAHRCT-COOH | 19 | 5xmore |
| Ac-V4(Bpa)/H9A-OH | Ac-ICV(Bpa)QDWGAHRCT-COOH | 20 | 49xmore |
| Ac-V4(Bpa)/H9A | Ac-ICV(Bpa)QDWGAHRCT-CONH2 | 21 | 86xmore |
| Ac-V4(Bta)/H9A-OH | Ac-ICV(Bta)QDWGAHRCT-COOH | 22 | 65xmore |
| Ac-V4(Bta)/H9A | Ac-ICV(Bta)QDWGAHRCT-CONH2 | 23 | 64xmore |
| Ac-V4W/H9(2-Abu) | Ac-ICVWQDWG(2-Abu)HRCT-CONH2 | 24 | 64xmore |
| +G/V4W/H9A+AN-OH | H-GICVWQDWGAHRCTAN-COOH | 25 | 38xmore |
| Ac-V4(5fW)/H9A | Ac-ICV(5fW)QDWGAHRCT-CONH₂ | 26 | 31xmore |
| Ac-V4(5-MeW)/H9A | Ac-ICV(5-methyl-W)QDWGAHRCT-CONH₂ | 27 | 67xmore |
| Ac-V4(1-MeW)/H9A | Ac-ICV(1-methyl-W)QDWGAHRCT-CONH₂ | 28 | 264xmore |
| Ac-V4W/W7(5fW)/H9A | Ac-ICVWQD(5fW)GAHRCT-CONH₂ | 29 | 121xmore |
| Ac-V4(5fW)/W7(5fW)/H9A | Ac-ICV(5fW)QD(5fW)GAHRCT-CONH₂ | 30 | NA |
| Ac-V4(5-MeW)/W7(5fW)H9A | Ac-ICV(5-methyl-W)QD(5fW)GAHRCT-CONH₂ | 31 | NA |
| Ac-V4(1MeW)/W7(5fW)/H9A | Ac-ICV(1-methyl-W)QD(5fW)GAHRCT-CONH₂ | 32 | 264xmore |
| +G/V4(6fW)/W7(6fW)H9A+N-OH | H-GICV(6fW)QD(6fW)GAHRCTN-COOH | 33 | 126xmore |
| Ac-V4(1-formyl-W)/H9A | Ac-ICV(1-formyl-W)QDWGAHRCT-CONH₂ | 34 | 264xmore |
| Ac-V4(5-methoxy-W)/H9A | Ac-ICV(1-methyoxy-W)QDWGAHRCT-CONH₂ | 35 | 76xmore |
| G/V4(5f-W)/W7(5fW)/H9A+N-OH | H-GICV(5fW)QD(5fW)GAHRCTN-COOH | 36 | 112xmore |

NA = not available

In certain embodiments of the compositions and methods of the disclosure, the compstatin analog has a sequence selected from sequences 9-36. In certain embodiments, the compstatin analog has a sequence of SEQ ID NO: 28. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in Table 1 contains at least one N-methyl isoleucine, e.g., at the position corresponding to position 13 of compstatin. For example, a Thr at or near the C-terminal end of a peptide whose sequence is listed in Table 1 or any other compstatin analog sequence may be replaced by N-methyl Ile. As will be appreciated, in some embodiments the N-methylated amino acids comprise N-methyl Gly at position 8 and N-methyl Ile at position 13. In some embodiments, a compstatin analog comprises an isoleucine at position 3 of SEQ ID NO:8, either instead of or in addition to one or more substitutions described herein. Additional compstatin analogs are described in, e.g., WO2019/166411.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3rd ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, CA, which also provides details of appropriate conjugation procedures.

In some embodiments, a compstatin analog of any of SEQ ID NOs: 2-36, is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of a compstatin analog described herein (e.g., a compstatin analog comprising any one of SEQ ID NOs: 9-36).

In some embodiments, the Lys residue is separated from the cyclic portion of the compstatin analog by a rigid or flexible spacer. The spacer may, for example, comprise a substituted or unsubstituted, saturated or unsaturated alkyl chain, oligo(ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the compstatin analogs. In one embodiment, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In another embodiment, short chains are prepolymerized, functionalized, and then a mixture of short chains of different composition are assembled into longer polymers.

In some embodiments a compstatin analog comprises a moiety such as a polyethylene glycol (PEG) chain or other polymer(s) that, e.g., stabilize the compound, increase its lifetime in the body, increase its solubility, decrease its immunogenicity, and/or increase its resistance to degradation. Without limiting the disclosure in any way, such a moiety may be referred to herein as a “clearance reducing moiety” (CRM). In some embodiments in which a compstatin analog comprises a PEG, e.g., of at least about 10 kD in size, the compound may be referred to as a PEGylated compstatin analog.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In some embodiments, multiple (CH₂CH₂O)ₙ moieties are provided as a branched structure. The branches may be attached to a linear polymer backbone (e.g., as a comb-shaped structure) or may emanate from one or more central core groups, e.g., as a star structure. In some embodiments, a branched molecule has 3 to 10 (CH₂CH₂O)ₙ chains. In some embodiments, a branched molecule has 4 to 8 (CH₂CH₂O)ₙ chains. In some embodiments, a branched molecule has 10, 9, 8, 7, 6, 5, 4, or 3 (CH₂CH₂O)ₙ chains. In some embodiments, a star-shaped molecule has 10-100, 10-50, 10-30, or 10-20 (CH₂CH₂O)ₙ chains emanating from a central core group. In some embodiments a PEGylated compstatin analog thus may comprise, e.g., 3-10 compstatin analog moieties, e.g., 4-8 compstatin analog moieties, each attached to a (CH₂CH₂O)n chain via a functional group at the end of the chain. In some embodiments a PEGylated compstatin analog may comprise, e.g., 10-100 compstatin analog moieties, each attached to a (CH₂CH₂O)n chain via a functional group at the end of the chain. In some embodiments, branches (sometimes referred to as “arms”) of a branched or star-shaped PEG contain about the same number of (CH₂CH₂O) moieties. In some embodiments, at least some of the branch lengths may differ. It will be understood that in some embodiments one or more (CH₂CH₂O)n chains does not have a compstatin analog moiety attached thereto. In some embodiments at least about 30%, 40%, 50%, 60%, 70%, 80%, 90%, 95%, or 100% of the chains has a compstatin analog moiety attached thereto.

In general and compounds depicted herein, a polyethylene glycol moiety is drawn with the oxygen atom on the right side of the repeating unit or the left side of the repeating unit. In cases where only one orientation is drawn, the present disclosure encompasses both orientations (i.e., (CH₂CH₂O)n and (OCH₂CH₂)ₙ) of polyethylene glycol moieties for a given compound or genus, or in cases where a compound or genus contains multiple polyethylene glycol moieties, all combinations of orientations are encompasses by the present disclosure.

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the —(CH₂CH₂O)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula A:

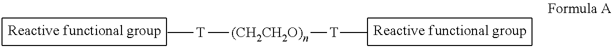

wherein each T and “Reactive functional group” is independently as defined below, and described in classes and subclasses herein, and n is as defined above and described in classes and subclasses herein.
Each T is independently a covalent bond or a C₁₋₁₂ straight or branched, hydrocarbon chain wherein one or more carbon units of T are optionally and independently replaced by —O—, —S—, —N(R^{x})—, —C(O)—, —C(O)O—, —OC(O)—, —N(R^{x})C(O)—, —C(O)N(R^{x})—, —S(O)—, —S(O)₂—, —N(R^{x})SO₂—, or —SO₂N(R^{x})—; and
each R^{x} is independently hydrogen or C₁₋₆ aliphatic.
The Reactive functional group has the structure —COO—NHS.

Exemplary bifunctional PEGs of formula A include:

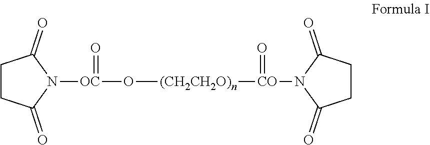

In some embodiments, a functional group (for example, an amine, hydroxyl, or thiol group) on a compstatin analog is reacted with a PEG-containing compound having a “reactive functional group” as described herein, to generate such conjugates. By way of example, Formula I can form compstatin analog conjugates having the structure:

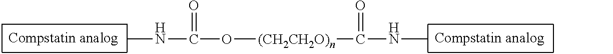

wherein,

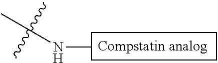 [SMILES: *NC]

represents the attachment point of an amine group on a compstatin analog. In certain embodiments, an amine group is a lysine side chain group.

In certain embodiments, the PEG component of such conjugates has an average molecular weight of about 5 kD, about 10 kD, about 15 kD, about 20 kD, about 30 kD, or about 40 kD. In certain embodiments, the PEG component of such conjugates has an average molecular weight of about 40 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

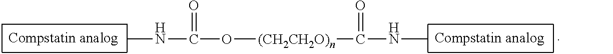

In some embodiments of formulae and embodiments described herein,

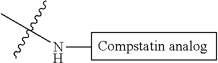 [SMILES: *NC]

represents point of attachment of a lysine side chain group in a compstatin analog having the structure:

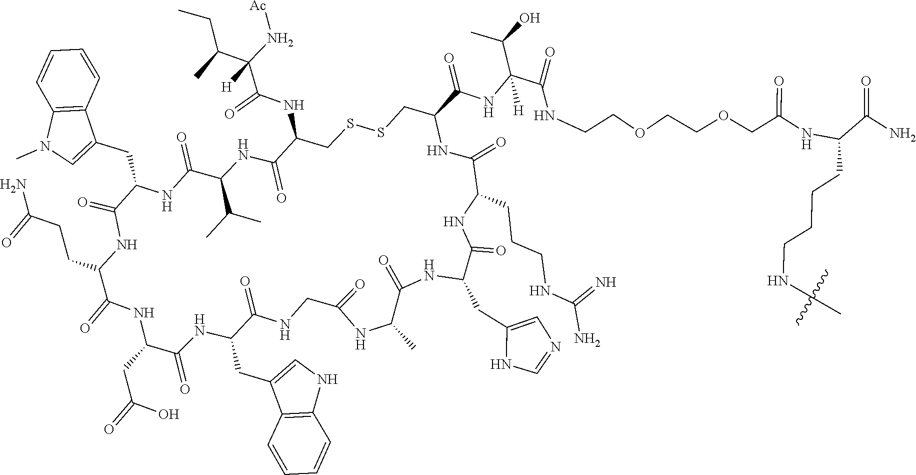

wherein the symbol “ [symbol] ” denotes the point of attachment of a chemical moiety to the remainder of a molecule or chemical formula.

PEGs comprising one or more reactive functional groups may, in some embodiments, be obtained from, e.g., NOF America Corp. White Plains, NY or BOC Sciences 45-16 Ramsey Road Shirley, NY 11967, USA, among others, or may be prepared using methods known in the art.

In some embodiments, a linker is used to connect a compstatin analog described herein and a PEG described herein. Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)nCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the α-amino group of an amino acid residue. In some embodiments, an amine group is the amine group of the lysine side chain. In some embodiments, a compstatin analog is conjugated to a PEG moiety through the amino group of a lysine side chain (ε-amino group) via a linker having the structure of NH₂(CH₂CH₂O)nCH₂C(═O)OH, wherein n is 1 to 1000. In some embodiments, a compstatin analog is conjugated to the PEG moiety through the amino group of a lysine side chain via an AEEAc linker. In some embodiments, the NH₂(CH₂CH₂O)nCH₂C(═O)OH linker introduces a —NH(CH₂CH₂O)nCH₂C(═O)— moiety on a compstatin lysine side chain after conjugation. In some embodiments, the AEEAc linker introduces a —NH(CH₂CH₂O)₂CH₂C(═O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the α-amino group of a lysine residue. In some embodiments, the C-terminus of a compstatin analog is connected to the amino group of AEEAc, the C-terminus of AEEAc is connected to the α-amino group of the lysine residue, and a PEG moiety is conjugated through the ε-amino group of said lysine residue. In some embodiments, the C-terminus of the lysine residue is modified. In some embodiments, the C-terminus of the lysine residue is modified by amidation. In some embodiments, the N-terminus of a compstatin analog is modified. In some embodiments, the N-terminus of a compstatin analog is acetylated.

In certain embodiments a compstatin analog may be represented as M-AEEAc-Lys-B₂, wherein B₂ is a blocking moiety, e.g., NH₂, M represents any of SEQ ID NOs: 9-36, with the proviso that the C-terminal amino acid of any of SEQ ID NOs: 9-36 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

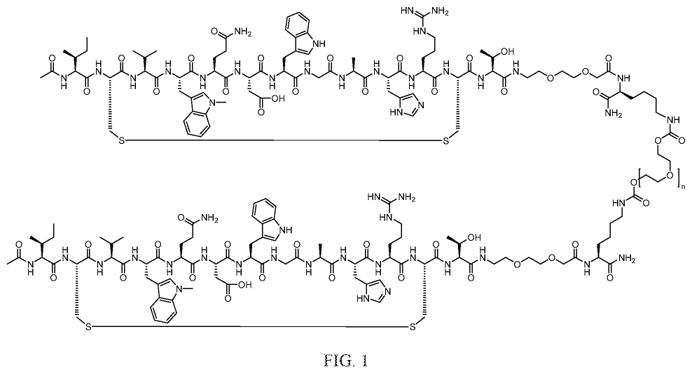

With regard to any of the above formulae and structures, it is to be understood that embodiments in which the compstatin analog component comprises any compstatin analog described herein, e.g., any compstatin analog of SEQ ID NOs; 9-36 are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted in FIG. 1. It will be understood that the PEG moiety may have a variety of different molecular weights or average molecular weights in various embodiments, as described herein. In certain embodiments, a compstatin analog is pegcetacoplan (“APL-2”), having the structure of the compound of FIG. 1 with n of about 800 to about 1100 and a PEG having an average molecular weight of about 40 kD. Pegcetacoplan is also referred to as Poly(oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-N⁶-carboxy-L-lysinamide cyclic (2->12)-(disulfide); or O,O′-bis[(S²,S¹²-cyclo{N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-L-lysinamide})-N^{6,15}-carbonyl]polyethylene glycol (n=800-1100). In certain embodiments, a compstatin analog has the structure of the compound of FIG. 1 with a PEG having an average molecular weight of about 10 kD. In certain embodiments, a compstatin analog has the structure of the compound of FIG. 1 with a PEG having an average molecular weight of about 20 kD. Additional compstatin analogs are described in, e.g., WO 2012/155107 and WO 2014/078731.

### IV. Complement-Mediated Eye Disorders

In some embodiments, a compstatin analog described herein is introduced into the eye of a subject for treatment of an eye disorder such as age-related macular degeneration (AMD). For example, a compstatin analog may be introduced into the vitreous cavity (e.g., by intravitreal injection), for treatment of a subject suffering from or at risk of AMD. In some embodiments the AMD is neovascular (wet) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with a compstatin analog as compared with control (e.g., patients given a sham injection). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and neovascular AMD. In some embodiments an eye has GA but not wet AMD. In some embodiments, a subject has one eye with GA and one eye without GA. In some embodiments, a subject has one eye with GA and one eye without wet AMD. In some embodiments, a subject has one eye with GA and one eye with wet AMD. In some embodiments, a subject has one eye having both GA and neovascular AMD and one eye without wet AMD. In some embodiments, a subject has one eye having both GA and neovascular AMD and one eye with wet AMD. In some embodiments, both eyes of a subject have both GA and neovascular AMD.

Geographic atrophy (GA) is a late-stage disease manifestation in nonneovascular age-related macular degeneration (AMD) that progresses to severe central vision loss. Geographic atrophy has traditionally been defined on color fundus photography (CFP) as a sharply delineated circular or oval area of hypopigmentation or depigmentation in which choroidal vessels are visible. The size requirement for GA varies with the different studies, ranging from one eighth to one fourth of a disc area (corresponding roughly to 175 μm and 430 μm in diameter, respectively) on CFP (see, e.g., Bird et al., Surv. Ophthalmol. 39:367-374 (1995) and Schmitz-Valckenberg, Ophthalmologica 237:11-20 (2017)). Geographic atrophy was retained as a term for a late stage of AMD in the 2013 Beckman CFP classification of AMD (see Ferris et al., Ophthalmology 120:844-851 (2013)). As a clinical trial end point, the ability to slow GA expansion using a novel therapy has been approved by regulatory authorities, in which atrophy can be defined and quantified by fundus autofluorescence (FAF) (Csaky et al., Invest. Ophthalmol. Vis. Sci. 58:3456-3463 (2017); Holz et al., Ophthalmology 121:1079-1091 (2014); Schmitz-Valckenberg et al., Ophthalmology 123:361-368 (2016)). Recent trials aiming to slow progression of atrophy were required to enroll eyes with established regions of GA that could be measured reliably by a reading center, usually measuring at least 0.5 to 1.0 disc areas (1.25-2.5 mm²) (Cheng et al., Ophthalmol. Retina 2:518-525 (2018); Holz et al., JAMA Ophthalmol. 136:666-677 (2018); Rosenfeld et al., Ophthalmology 125:1556-1567 (2018)). In some embodiments, GA is assessed using FAF. In some embodiments, an eye (e.g. an eye of a subject with GA) has at least one iRORA lesion located beyond a 500-μm (i.e., 500 micron) perimeter from the GA border.

Drusen are localized extracellular deposits of lipoproteinaceous material that accumulate between the retinal pigment epithelium (RPE) and the capillary network in the choroid (choriocapillaris), typically between the RPE and Bruch's membrane (a multilayered extracellular matrix complex that separates the RPE from the choriocapillaris). It will be appreciated that the term “druse” is sometimes used in the art to refer to a single such deposit (i.e., as a singular referent) while “drusen” is sometimes used in the art to refer to multiple such deposits (i.e., as a plural referent). As used herein, the term “drusen” should be understood to encompass a single “druse” or multiple “drusen” in various embodiments unless indicated to the contrary or clearly evident from the context. Also, reference to “a druse” should be understood to encompass reference to a single “druse” or multiple “drusen” in various embodiments unless indicated to the contrary or clearly evident from the context. Drusen are a clinical hallmark of AMD and are typically the earliest clinical finding in AMD. The presence, location, size, and number of drusen are factors used in the art for classifying AMD into stages and monitoring its progression. However, a few small drusen are commonly observed in the eyes of people over 40 years of age, most of whom do not go on to develop AMD. Drusen can be detected, assessed, and/or classified according to known methods, e.g., imaging, scanning laser ophthalmoscopy, and optical coherence tomography (OCT) (e.g., spectral domain optical coherence tomography (SD-OCT)). Additional methods are described in, e.g., WO2014/028861.

In some embodiments early, intermediate, or advanced AMD are defined in accordance with the classification scheme used in the Age-Related Eye Diseases Study (AREDS) (The Age-Related Eye Disease Study Research Group. A randomized, placebo-controlled, clinical trial of high-dose supplementation with vitamins C and E, beta carotene, and zinc for age-related macular degeneration and vision loss: AREDS report number 8. Arch Ophthalmol 2001; 119:1417-36). The classification of AMD from the AREDS is summarized as follows:
No AMD (AREDS category 1) is characterized by no or few small drusen (<63 microns in diameter).
Early AMD (AREDS category 2) is characterized by presence of a combination of multiple small drusen, few intermediate drusen (63 to 124 microns in diameter), or RPE abnormalities.
Intermediate AMD (AREDS category 3) is characterized by presence of extensive intermediate drusen, at least one large druse (at least 125 microns in diameter), or geographic atrophy not involving the center of the macula.
Advanced AMD (AREDS category 4) is characterized by geographic atrophy involving the center of the macula and/or neovascular macular degeneration. Neovascular macular degeneration is typically associated with manifestations of CNV and/or retinal or RPE detachment associated with subretinal serous fluid, exudates, and/or blood. Other manifestations of neovascular AMD may include retinal hard exudates, subretinal and sub-RPE fibrovascular proliferation, and/or disciform scar.

Other classification schemes, or modified forms of the AREDS scheme, may be used. For example, in some embodiments an eye exhibiting geographic atrophy is considered to have advanced AMD whether or not the macula is involved. In some embodiments, AMD is classified as described in, e.g., Ferris et al., Ophthalmology 120:844-851 (2013). In some embodiments, AMD is classified as described in the ICD10 classification system.

In some embodiments, a subject is treated with a compstatin analog described herein to slow or halt progression (e.g., in one or both eyes) (i) from incomplete retinal pigment epithelium and outer retinal atrophy (“iRORA”) to complete retinal pigment epithelium and outer retinal atrophy (“cRORA”), and/or (ii) from drusen (e.g., large drusen) to iRORA and/or to cRORA. Features of iRORA and cRORA are known in the art, and can be identified using multimodal imaging, such as OCT (e.g., SD-OCT or swept-source OCT) (see, e.g., Sadda et al., Ophthalmology 125:537-548 (2018); Guymer et al., Ophthalmology 127:394-409 (2020)). In some embodiments, OCT is B-scan OCT. For example, in the context of AMD with conventional drusen, iRORA is defined on OCT by the following criteria (in the absence of signs of a retinal pigment epithelium (RPE) tear): (1) a region of signal hypertransmission into the choroid, (2) a corresponding zone of attenuation or disruption of the RPE, with or without persistence of basal laminar deposits, (3) evidence of overlying photoreceptor degeneration, that is, subsidence of the inner nuclear layer (INL) and outer plexiform layer (OPL), presence of a hyporeflective wedge in the Henle fiber layer (HFL), thinning of the outer nuclear layer (ONL), disruption of the external limiting membrane (ELM), or disintegrity of the ellipsoid zone (EZ), and (4) when these criteria do not meet the definition of cRORA. cRORA is defined on OCT by the following criteria (in the absence of signs of an RPE tear): (1) a region of hypertransmission of at least 250 μm in diameter, (2) a zone of attenuation or disruption of the RPE of at least 250 μm in diameter, and (3) evidence of overlying photoreceptor degeneration. In some embodiments, a subject is treated with a compstatin analog described herein to slow or halt progression (e.g., in an area of one or both eyes without GA) (i) from incomplete retinal pigment epithelium and outer retinal atrophy (“iRORA”) to complete retinal pigment epithelium and outer retinal atrophy (“cRORA”), and/or (ii) from drusen (e.g., large drusen) to iRORA and/or to cRORA.

In some embodiments, a subject exhibits nascent GA (nGA) in one or both eyes. In some embodiments, nGA can be defined as presence of iRORA in the absence of current and/or prior macular neovascularization. In some embodiments, a subject exhibits GA in one or both eyes. In some embodiments, a subject exhibits GA in one or both eyes in absence of current and/or past macular neovascularization in one or both eyes. In some embodiments, a subject exhibits GA in one or both eyes in presence of current and/or past macular neovascularization in one or both eyes.

In some embodiments, a subject (e.g., a subject having or suffering from AMD in one or both eyes) exhibiting one or more drusen (e.g., greater than 20, 30, 35, 40, 45, 50, 55, 60, 70, or 80 microns in size) in one or both eyes is treated with a compstatin analog described herein such that progression to iRORA in one or both eyes is slowed or halted. For example, a compstatin analog described herein (e.g., pegcetacoplan) is administered as a dosing regimen monthly or every other month for at least 6 months, 12 months, 18 months, 24 months, 30 months, 36 months, or longer, and the subject does not develop iRORA in one or both eyes for at least 3 months, 6 months, 12 months, 18 months, 24 months, 30 months, 36 months, or longer, following the start of administering the dosing regimen. In some embodiments, a compstatin analog described herein (e.g., pegcetacoplan) is administered as a dosing regimen monthly or every other month for at least 6 months, 12 months, 18 months, 24 months, 30 months, 36 months, or longer, and the subject exhibits a slower progression to iRORA, e.g., relative to a control (e.g., a control eye of a control subject having or suffering from AMD) which is not treated with the compstatin analog (e.g., pegcetacoplan) and which exhibits one or more drusen of comparable size to the treated eye of the treated subject. In some embodiments, one or both treated eyes of a treated subject exhibits iRORA at a delayed period, relative to the control, that is at least 1 month, 2 months, 3 months, 4 months, 5 months, 6 months, 12 months, 18 months, 24 months, 30 months, 36 months, or longer than the time a control eye of a control subject exhibits iRORA.

In some embodiments, a subject (e.g., a subject having or suffering from AMD in one or both eyes) exhibiting one or more drusen (e.g., greater than 20, 30, 35, 40, 45, 50, 55, 60, 70, or 80 microns in size) in one or both eyes is treated with a compstatin analog described herein, and progression to cRORA is slowed or halted. For example, a compstatin analog described herein (e.g., pegcetacoplan) is administered as a dosing regimen monthly or every other month for at least 6 months, 12 months, 18 months, 24 months, 30 months, 36 months, or longer, and the subject does not develop cRORA in one or both eyes for at least 3 months, 6 months, 12 months, 18 months, 24 months, 30 months, 36 months, or longer, following the start of administering the dosing regimen. In some embodiments, a compstatin analog described herein (e.g., pegcetacoplan) is administered as a dosing regimen monthly or every other month for at least 6 months, 12 months, 18 months, 24 months, 30 months, 36 months, or longer, and the subject exhibits a slower progression to cRORA, e.g., relative to a control (e.g., a control eye of a control subject having or suffering from AMD) which is not treated with the compstatin analog (e.g., pegcetacoplan) and which exhibits one or more drusen of comparable size to the treated eye of the treated subject. In some embodiments, one or both treated eyes of a treated subject exhibits cRORA at a delayed period, relative to the control, that is at least 1 month, 2 months, 3 months, 4 months, 5 months, 6 months, 12 months, 18 months, 24 months, 30 months, 36 months, or longer than the time a control eye of a control subject exhibits cRORA.

In some embodiments, a subject exhibiting iRORA in one or both eyes is treated with a compstatin analog described herein, and progression to cRORA in one or both eyes is slowed or halted. For example, a compstatin analog described herein (e.g., pegcetacoplan) is administered as a dosing regimen monthly or every other month for at least 6 months, 12 months, 18 months, 24 months, 30 months, 36 months, or longer, and the subject does not develop cRORA in one or both eyes for at least 3 months, 6 months, 12 months, 18 months, 24 months, 30 months, 36 months, or longer, following the start of administering the dosing regimen. In some embodiments, a compstatin analog described herein (e.g., pegcetacoplan) is administered as a dosing regimen monthly or every other month for at least 6 months, 12 months, 18 months, 24 months, 30 months, 36 months, or longer, and the subject exhibits a slower progression to cRORA, relative to a control (e.g., a control eye of a control subject having or suffering from AMD) which is not treated with the compstatin analog (e.g., pegcetacoplan) and which exhibits iRORA comparable to the treated eye of the treated subject. In some embodiments, one or both treated eyes of a treated subject exhibits cRORA at a delayed period, relative to the control, that is at least 1 month, 2 months, 3 months, 4 months, 5 months, 6 months, 12 months, 18 months, 24 months, 30 months, 36 months or longer than the time a control eye of a control subject exhibits cRORA. In some embodiments, one eye exhibits iRORA and one eye exhibits GA. In some embodiments, one eye exhibits iRORA and one eye does not exhibit GA. In some embodiments, both eyes exhibit iRORA. In some embodiments, one eye exhibits iRORA and does not exhibit GA. In some embodiments, one eye exhibits iRORA and also exhibits GA. In some embodiments, one eye exhibits iRORA and does not exhibit GA, and one eye exhibits GA. In some embodiments, one eye exhibits iRORA and does not exhibit GA, and one eye does not exhibit GA. In some embodiments, one eye exhibits iRORA and exhibits GA, and one eye does not exhibit GA. In some embodiments, one eye exhibits iRORA and exhibits GA, and one eye exhibits GA. In some embodiments, both eyes exhibit iRORA and do not exhibit GA. In some embodiments, both eyes exhibit iRORA and both eyes exhibit GA.

### V. Compositions and Administration

The disclosure provides and/or utilizes a variety of compositions comprising a compstatin analog. In various embodiments, a composition can have any feature or combination of features discussed herein so long as they are not mutually exclusive. The invention provides embodiments of such compositions, and methods of use thereof, in which the compstatin analog is any compstatin analog, e.g., an analog described herein.

Suitable preparations, e.g., substantially pure preparations of a compstatin analog or other active agent, may be combined with pharmaceutically acceptable carriers or vehicles, etc., to produce an appropriate pharmaceutical composition. The term “pharmaceutically acceptable carrier or vehicle” refers to a non-toxic carrier or vehicle that does not destroy the pharmacological activity of the compound with which it is formulated. One of skill in the art will understand that a carrier or vehicle is “non-toxic” if it is compatible with administration to a subject in an amount appropriate to deliver the compound without causing undue toxicity. Pharmaceutically acceptable carriers or vehicles that may be used in the compositions of this invention include, but are not limited to, water, physiological saline, Ringer's solution, sodium acetate or potassium acetate solution, 5% dextrose, and the like. The composition may include other components as appropriate for the formulation desired, e.g., as discussed herein. Supplementary active compounds, e.g., compounds independently useful for treating a subject suffering from a complement-mediated disorder, can also be incorporated into the compositions. The invention provides such pharmaceutical compositions comprising a compstatin analog and, optionally, a second active agent useful for treating a subject suffering from AMD.

In some embodiments, the invention provides a pharmaceutically acceptable composition suitable for administration to humans, packaged together with a label approved by a government agency responsible for regulating pharmaceutical agents, e.g., the U.S. Food & Drug Administration. In some embodiments, the invention provides a pharmaceutical kit or pack comprising: (a) a pharmaceutically acceptable compstatin analog in solid form; (b) a pharmaceutically acceptable carrier or vehicle. In some embodiments the solid form is a lyophilized form. In some embodiments the solid form is a powder form, e.g., a lyophilized powder. Optionally the kit or pack contains instructions for dissolving the compstatin analog in the carrier. In some embodiments a pharmaceutical kit or pack is provided. In some embodiments the pack or kit comprises sufficient amount of pharmaceutical composition for at least 1 dose, e.g., between 1 and 200 doses or any intervening number or subrange. In some embodiments a kit or pack comprises (i) a first container containing sufficient compstatin analog for one or more doses; (ii) a second container comprising a pharmaceutically acceptable carrier to be combined with the contents of the first vessel to produce a composition suitable for administration to a subject by, e.g., intravitreal (IVT) injection. In some embodiments a pharmaceutical pack or kit comprises one or more needles and, optionally, one or more syringes. In some embodiments at least one prefilled syringe is provided (e.g., between 1 and 200 or any intervening number of subrange). In some embodiments one or more unit dosage forms or premeasured aliquots are provided. In some embodiments instructions for administration are provided.

A pharmaceutical composition can be administered to a subject by any suitable route of administration including, but not limited to, intravitreally, intravenous, intramuscular, subcutaneously, by inhalation, by nasal delivery, intrathecally, intracranially, intraarterially, orally, rectally, transdermally, intradermally, subdermally, etc. It will be understood that “administration” encompasses directly administering a compound or composition to a subject, instructing a third party to administer a compound or composition to a subject, prescribing or suggesting a compound or composition to a subject, and, as appropriate, other means of making a compound or composition available to a subject.

Pharmaceutical compositions suitable for injectable use (e.g., intravitreal administration) typically include sterile aqueous solutions (where water soluble) or dispersions and sterile powders for the extemporaneous preparation of sterile injectable solutions or dispersion. Sterile solutions can be prepared by incorporating the compound in the required amount in an appropriate solvent, optionally with one or a combination of ingredients such as buffers such as acetates, citrates, lactates or phosphates; agents for the adjustment of tonicity such as sodium chloride or dextrose, mannitol, sorbitol, or trehalose; antibacterial agents such as benzyl alcohol or methyl parabens; antioxidants such as ascorbic acid, glutathione, or sodium bisulfite; chelating agents such as ethylenediaminetetraacetic acid; and other suitable ingredients etc., as desired, followed by filter-based sterilization. One of skill in the art will be aware of numerous physiologically acceptable compounds that may be included in a pharmaceutical composition. Other useful compounds include, for example, carbohydrates, such as glucose, sucrose, lactose, or trehalose; dextrans; amino acids such as glycine; polyols such as mannitol or sorbitol, e.g., sugar alcohols (compounds having the general formula HOCH₂(CHOH)ₙCH₂OH). These compounds may, for example, serve as bulking agents and/or stabilizers, e.g., in a powder and/or when part of the manufacture or storage process involves lyophilization. In some aspects, such compound(s) may serve as osmolality modifiers. Surfactant(s) such as Tween-80, Pluronic-F108/F68, deoxycholic acid, phosphatidylcholine, etc., may be included in a composition, e.g., to increase solubility or to provide microemulsion to deliver hydrophobic drugs. pH can be adjusted with acids or bases, such as hydrochloric acid or sodium hydroxide, if desired. Preferably solutions for injection are sterile and acceptably free of endotoxin.

In certain embodiments a composition comprising a compstatin analog, or a composition to which a compstatin analog is to be added, comprises one or more non-reducing sugars or sugar alcohols, e.g., trehalose, mannitol, or sorbitol. In certain embodiments a composition comprising a compstatin analog, or a composition to which a compstatin analog is to be added, comprises one or more reducing sugars, e.g., dextrose. One of ordinary skill in the art appreciates that a reducing sugar is a sugar that is capable of acting as a reducing agent, typically because it has a free aldehyde group or a free ketone group, e.g., when it assumes an open-chain form.

In some embodiments, compositions provided by the present disclosure include a compstatin analog and do not include a reducing sugar, e.g., dextrose, as a component. In some embodiments a composition comprising a compstatin analog, or to which a compstatin analog is to be added, is free or substantially free of one or more reducing sugars, e.g., dextrose. In certain embodiments a composition comprising a compstatin analog, or to which a compstatin analog is to be added, is free or substantially free of reducing sugars. A composition is “substantially free” of a particular substance or substances if the composition contains no more than 0.1% weight/volume (w/v) of that substance or substances, e.g., no more than 0.01% w/v, e.g., no more than 0.001% w/v, of that substance or substances.

In some embodiments of any of the aspects described herein, a composition described herein comprises a compstatin analog described herein and water. In some embodiments of any of the aspects described herein, a composition described herein comprises a compstatin analog described herein, water, and one or both of a tonicity adjusting agent and a buffer substance. In some embodiments of any of the aspects described herein, a composition described herein consists essentially of or consists of a compstatin analog described herein and one or more specified components, e.g., a buffer substance and/or a tonicity adjusting agent. In some embodiments, such a composition consists of such substances in water as a pharmaceutically acceptable carrier. In some embodiments of any of the aspects described herein, a composition comprising a compstatin analog described herein has a pH as described herein.

As described herein, in some embodiments, a compstatin analog in dry powder form (e.g., having been synthesized, optionally in water or other aqueous solvent, and dried, e.g., by lyophilization), may be reconstituted, for example in water or an aqueous buffer, prior to administration in a suitable volume of a buffered aqueous solution containing one or more excipients (e.g., sodium chloride, dextrose, mannitol, sorbitol, trehalose, or a combination thereof) to form a solution containing a selected concentration of the compstatin analog and having a selected osmolality. In some embodiments a compstatin analog may be or have been dried (e.g., lyophilized) in a buffered solution containing one or more excipients (e.g., dextrose, mannitol, sorbitol, or trehalose) in an appropriate amount so that reconstitution of the lyophilized material in an appropriate volume of water will yield a solution having a selected concentration of the compstatin analog and a desired osmolality. A selected concentration may be any of the concentrations described herein. A selected osmolality may be any of the osmolalities described herein. In certain embodiments the composition (e.g., the composition prior to drying or the composition after resuspending) comprises one or more excipients in an amount such that the composition is approximately isotonic with respect to normal human plasma. In some aspects, a composition is considered “isotonic with respect to normal human plasma” (also referred to simply as “isotonic”) if the concentration of solutes that cannot freely diffuse across a plasma membrane of a normal human cell, e.g., a normal human red blood cell or an epithelial cell, is between 260 mOsm/kg and 320 mOsm/kg, e.g., between 280 mOsm/kg and 300 mOsm/kg, e.g., 285 mOsm/kg and 295 mOsm/kg. In some embodiments the one or more excipients is/are selected from the group consisting of dextrose, sodium chloride, mannitol, sorbitol, and trehalose.

Those skilled in the art, reading the present disclosure, will appreciate that, in accordance with standard practice in the field, a container containing a particular volume, as described herein my include an additional volume sufficient to permit the designated particular volume (e.g., unit dose) to be withdrawn from the container for administration.

In some embodiments the composition is either lyophilized or kept refrigerated or frozen until shortly before administration, at which time it is reconstituted (if lyophilized) or thawed (if frozen). The composition may be brought to room temperature prior to administration.

In some embodiments a composition comprising a compstatin analog described herein and a pharmaceutically acceptable carrier has a pH of between 6.5 and 7.5, e.g., between 6.8 and 7.2, e.g., 7.0. In some embodiments, a composition comprising a compstatin analog and a pharmaceutically acceptable carrier has a pH of between 6.0 and 6.5, between 5.5 and 6.0, or between 5.0 and 5.5. In certain embodiments a yet lower pH, e.g., between 4.5 and 5.0 may be used. In particular embodiments, for example, a composition has a pH between 4.6 and 5.4, e.g., between 4.8 and 5.2, between 4.9 and 5.1, e.g., 5.0. In some embodiments the composition further comprises one or more pharmaceutically acceptable buffer substances appropriate to maintain the pH within a selected range (e.g., any of the afore-mentioned ranges). Suitable buffer substances are described herein (e.g., acetates, citrates, lactates or phosphates). In some embodiments the composition additionally or alternately comprises a salt, e.g., any of the pharmaceutically acceptable salts described herein. In certain embodiments the buffer substance comprises sodium acetate. In certain embodiments the buffer substance, e.g., sodium acetate, is present at a concentration between 10 mM and 50 mM, e.g., 10 mM-15 mM, 15 mM-20 mM, 20 mM-25 mM, 25 mM-30 mM, 30 mM-35 mM, 35 mM-40 mM, 40 mM-50 mM. In particular embodiments the buffer substance is present at a concentration of about 15 mM, about 17.5 mM, about 20 mM, about 22.5 mM, about 25 mM, about 27.5 mM, or about 30 mM. In certain embodiments the sodium acetate may be provided by including acetic acid and sodium acetate trihydrate in the solution. In certain particular embodiments, the composition comprises a phosphate.

In some aspects, described herein is a composition comprising a compstatin analog, wherein the composition has a pH of 6.5 or below (e.g., between 6.0 and 6.5, between 5.5 and 6.0, between 5.0 and 5.5, or between 4.5 and 5.0). In some aspects, it is contemplated to increase the stability and/or improve one or more other properties of a compstatin analog in a composition by reducing the pH of the composition to 6.5 or below (e.g., between 6.0 and 6.5, between 5.5 and 6.0, between 5.0 and 5.5, or between 4.5 and 5.0) or formulating the composition to have a pH of 6.5 or below (e.g., between 6.0 and 6.5, between 5.5 and 6.0, between 5.0 and 5.5, or between 4.5 and 5.0). In some embodiments the compstatin analog may comprise any compstatin analog described herein. Without wishing to be bound by any particular theory, in some embodiments a pH of 6.5 or below (e.g., between 6.0 and 6.5, between 5.5 and 6.0, between 5.0 and 5.5, or between 4.5 and 5.0), may be particularly useful to stabilize compstatin analogs that comprise a carbamate linkage, e.g., compounds wherein a compstatin analog moiety is linked to a clearance reducing moiety via a carbamate linkage, e.g., in pharmaceutical compositions comprising such a compstatin analog, which may be stored for prolonged periods. Alternatively or additionally, without wishing to be bound by any theory, in some embodiments a pH of 6.5 or below (e.g., between 6.0 and 6.5, between 5.5 and 6.0, between 5.0 and 5.5, or between 4.5 and 5.0), may be useful to stabilize compstatin analogs that comprise any compstatin analog moiety as described herein and/or to afford one or more other improvements to compositions comprising such compstatin analogs.

Generally, dispersions are prepared by incorporating the active compound into a sterile vehicle which contains a basic dispersion medium and appropriate other ingredients from those enumerated above. In the case of sterile powders for the preparation of sterile injectable solutions, methods of preparation can include vacuum drying and freeze-drying which yields a powder of the active ingredient plus any additional desired ingredient, e.g., from a previously sterile-filtered solution thereof.

It will be appreciated that the compstatin analog and/or additional active agent(s) can be provided as a pharmaceutically acceptable salt. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Also, pharmaceutically-acceptable salts can be prepared as alkaline metal or alkaline earth salts, such as sodium, potassium or calcium salts, if appropriate depending on the identity of the active agent.

It will be understood that the pharmaceutically acceptable carriers, compounds, and preparation methods mentioned herein are exemplary and non-limiting. See, e.g., Remington: The Science and Practice of Pharmacy. 21st Edition. Philadelphia, PA. Lippincott Williams & Wilkins, 2005, for additional discussion of pharmaceutically acceptable compounds and methods of preparing pharmaceutical compositions of various types.

A pharmaceutical composition can be administered in an amount effective to achieve a desired beneficial effect. In some embodiments, an effective amount is sufficient to provide one or more of the following benefits: (i) reduction in at least one symptom or sign of AMD; (ii) slowing or halting progression from iRORA to cRORA, and/or (iii) slowing or halting progression from drusen (e.g., large drusen) to iRORA and/or to cRORA. In some aspects, a beneficial effect is statistically significant and/or therapeutically meaningful within the judgement of one or ordinary skill in the art.

In some embodiments the compositions comprise 5% dextrose as a pharmaceutically acceptable carrier. However, any pharmaceutically acceptable carrier may be used. For example, in some embodiments a composition suitable for intravitreal administration of a compstatin analog, comprises one or more excipients selected from the group consisting of mannitol, sorbitol, trehalose, and sodium chloride, or combinations thereof.

In some embodiments a composition comprising a compstatin analog may have a selected osmolality. In some embodiments a composition comprising a compstatin analog, e.g., a composition for ocular, e.g., intravitreal, administration, has an osmolality of between 150 milliosmoles/kilogram (mOsm/kg) and 600 mOsm/kg, e.g., between 180 mOsm/kg and 500 mOsm/kg, e.g., between 200 mOsm/kg and 400 mOsm/kg.

In certain embodiments a composition comprising a compstatin analog has an osmolality between 250 mOsm/kg and 380 mOsm/kg, e.g., between 275 mOsm/kg and 350 mOsm/kg. In certain embodiments a composition comprising a compstatin analog has an osmolality of between 275 mOsm/kg and 285 mOsm/kg, between 285 mOsm/kg and 295 mOsm/kg, between 295 mOsm/kg and 305 mOsm/kg, or between 305 mOsm/kg and 315 mOsm/kg. In particular embodiments the osmolality of the composition is 300 mOsm/kg.

In certain embodiments a composition comprising a compstatin analog may be used for ocular administration, e.g., intravitreal administration.

In some embodiments the osmolality of a composition may be measured using a vapor pressure depression osmometer. In some embodiments the osmolality of a composition may be measured using a membrane osmometer. In some embodiments the osmolality of a composition may be measured using a freezing point depression osmometer.

In some aspects, described herein are various doses, dosing regimens, compositions, and methods useful for treating patients by IVT injection, e.g., in a manner that is acceptable to physicians and patients in terms of the time and pressure required to deliver a given volume of the composition through a needle of a given inner diameter or gauge number. Gauge number describes the outer diameter of a hollow needle, with a higher gauge number indicating a smaller outer diameter. Generally, needles having a higher gauge number may be preferred by patients and physicians as they may be associated with (or may be perceived to be associated with) less pain and/or tissue damage as compared with needles having a lower gauge number. Inner diameter depends on both outer diameter and wall thickness. For standard needles, the higher the gauge number, the smaller the inner diameter (e.g., a 27 gauge needle has a larger inner diameter than a 29 gauge needle, which in turn has a larger inner diameter than a 30 gauge needle).

In some embodiments a composition, e.g., a pharmaceutical composition, comprising a compstatin analog has a concentration of 150 mg/ml. In some embodiments a 27 gauge needle is used to administer a dose of a compstatin analog comprising a PEG having a molecular weight of about 40 kD at a concentration of up to 150 mg/ml, e.g., 75 mg/ml-90 mg/ml, 85 mg/ml-95 mg/ml, 90 mg/ml-100 mg/ml, 95 mg/ml-105 mg/ml 100 mg/ml-110 mg/ml, 105 mg/ml-115 mg/ml, 110 mg/ml-120 mg/ml, 115 mg/ml-125 mg/ml, 120 mg/ml-130 mg/ml, 125 mg/ml-135 mg/ml, 130 mg/ml-140 mg/ml, 135 mg/ml-145 mg/ml, 140 mg/ml-150 mg/ml, e.g., 100 mg/ml, 125 mg/ml, or 150 mg/ml. In some embodiments a composition comprising a compstatin analog (e.g., comprising a PEG having a molecular weight of about 40 kD) has a concentration of up to 150 mg/ml, e.g., 75 mg/ml-90 mg/ml, 85 mg/ml-95 mg/ml, 90 mg/ml-100 mg/ml, 95 mg/ml-105 mg/ml 100 mg/ml-110 mg/ml, 105 mg/ml-115 mg/ml, 110 mg/ml-120 mg/ml, 115 mg/ml-125 mg/ml, 120 mg/ml-130 mg/ml, 125 mg/ml-135 mg/ml, 130 mg/ml-140 mg/ml, 135 mg/ml-145 mg/ml, 140 mg/ml-150 mg/ml, e.g., 100 mg/ml, 125 mg/ml, or 150 mg/ml. In some embodiments a composition comprising a compstatin analog (e.g., comprising a PEG having a molecular weight of about 40 kD) has a concentration of 150 mg/ml-180 mg/ml. In some embodiments a composition comprising a compstatin analog (e.g., comprising a PEG having a molecular weight of about 40 kD) has a concentration of 180 mg/ml-200 mg/ml. In some embodiments a 27 gauge needle is used to administer a composition comprising a compstatin analog (e.g., comprising a PEG having a molecular weight of about 40 kD) at a concentration of 150 mg/ml-160 mg/ml, 160 mg/ml-170 mg/ml, 170 mg/ml-180 mg/ml, 180 mg/ml-190 mg/ml, or 190 mg/ml-200 mg/ml. Of course a needle with a lower gauge number (e.g., 25, 26 gauge) may alternately be used instead of a needle with a gauge number of 27 gauge (or higher).

In some embodiments a 25 gauge needle or a 26 gauge needle is used to administer a composition comprising a compstatin analog (e.g., comprising a PEG having a molecular weight of about 40 kD) at a concentration of 200 mg/ml, or more. In some embodiments a 25 gauge needle or a 26 gauge needle is used to administer a composition comprising a compstatin analog (e.g., comprising a PEG having a molecular weight of about 40 kD) at a concentration of 200 mg/ml-225 mg/ml or 225 mg/ml-250 mg/ml.

In some aspects, the present disclosure teaches particular utility of certain thin-walled needles for administration of a compstatin analog in accordance with the present invention. For example, in some embodiments a thin wall needle is used for intraocular (e.g., intravitreal) injection of a compstatin analog. Thin wall needles have identical outer diameters to standard needles but larger inner diameters for a given gauge. For example, a thin wall needle may have an internal diameter size that is the same as that of a standard needle of a gauge one to two numbers lower (e.g., a 29 gauge thin wall needle may have an internal diameter that is the same as that of a 27 gauge or 28 gauge standard needle but an outer diameter that is the same as that of a standard 29 gauge needle). An increase in internal diameter can result in a considerable increase in fluid flow for a given pressure and/or a considerable reduction in pressure needed to maintain a given flow. Lower pressure means that less injection force is needed to administer a composition of a given viscosity. In general, low injection force facilitates administration and is therefore typically a desirable feature. In some embodiments a thin wall needle has a given internal diameter that is uniform along the length of the needle. In some embodiments a thin wall needle has an internal diameter that varies along the length of the needle. For example, the diameter may be the same as that of a standard 29 gauge needle at one end of the needle and progress to the diameter of a standard 27 gauge needle at the other end. In some embodiments a microtapered needle may be used. In some embodiments a needle with a scalpel-like tip may be used. The length of the needle may vary. In some embodiments a short needle such as a 5 mm or 6 mm needle may be used. In some embodiments a needle having a length between 6 mm and 8 mm, or between 8 mm and 12 mm may be used. Suitable needles and syringes are available commercially, e.g., from Becton Dickinson and Company (BD), Terumo Corp., etc.

In some embodiments a composition having a given viscosity and/or concentration may be administered using a thin wall needle having a gauge that is one or two numbers higher than the gauge size that is preferably used when a standard needle is used to administer a composition of the same viscosity and/or concentration at a selected flow rate and/or with a selected injection force. For example, in some embodiments a composition that is preferably administered using a 25 gauge standard needle in order to attain a desired flow rate and/or injection force can be administered with such a flow rate and/or injection force using a 26 or 27 gauge thin wall needle. In some embodiments a composition that is preferably administered using a 27 gauge standard needle in order to attain a desired flow rate and/or injection force can be administered with such a flow rate and/or injection force using a 28 or 29 gauge thin wall needle. In some embodiments a composition that is preferably administered using a 29 gauge standard needle in order to attain a desired flow rate and/or injection force can be administered with such a flow rate and/or injection force using a 30 or 31 gauge thin wall needle. In some embodiments a 29 gauge thin wall needle is used to administer a composition comprising a compstatin analog (e.g., comprising a PEG having a molecular weight of about 40 kD) at a concentration of 150 mg/ml-160 mg/ml, 160 mg/ml-170 mg/ml, 170 mg/ml-180 mg/ml, 180 mg/ml-190 mg/ml, or 190 mg/ml-200 mg/ml. Of course a thin wall needle with a lower gauge number (e.g., 27, 28 gauge) may alternately be used instead of a thin wall needle with a gauge number of 29 gauge (or higher).

In some aspects, described herein are various doses, dosing regimens, compositions and methods of use for treating a patient in need of treatment for a complement-mediated eye disorder that affects the posterior segment of the eye, e.g., the retina, by intravitreal (IVT) administration of a compstatin analog. In some embodiments, the eye disorder is AMD, e.g., advanced AMD (geographic atrophy (GA) or neovascular AMD).

In some embodiments a dose for intravitreal injection of a compstatin analog (e.g., comprising two compstatin analog moieties and a PEG having a molecular weight of about 40 kD) is 5 mg-20 mg. In some embodiments the dose is 10 mg. In some embodiments the dose is 10 mg, 11 mg, 12 mg, 13 mg, 14 mg, 15 mg, 16 mg, 17 mg, 18 mg, 19 mg, or 20 mg. In certain particular embodiments the dose is 15 mg. In some embodiments, any of the afore-mentioned doses is administered by intravitreal injection in a volume of between 90 and 110 microliters, e.g., in a volume of 100 microliters. In some embodiments, any of the afore-mentioned doses is administered by intravitreal injection using a 27, 28, 29, or 30 gauge needle. In a Phase 1b clinical trial, it was found that doses of up to 20 mg (i.e., 5 mg, 10 mg, and 20 mg) in a volume of 100 microliters administered by intravitreal injection using a 29 gauge needle were well tolerated by patients with AMD. In certain embodiments a combination of dose and needle gauge that allows administration by IVT injection in 10 seconds or less is selected. In certain embodiments a combination of dose and needle that allows administration of the dose by IVT injection in 5-6 seconds, or less, is used. It was found that a dose of 15 mg in a volume of 100 microliters (150 mg/ml) results in a composition that has a favorable viscosity for administration by intravitreal injection in 5-6 seconds or less via a thin wall 27 gauge needle.

In certain embodiments a dose of a composition comprising a compstatin analog is administered by IVT injection in a volume greater than 100 microliters. For example, a volume of 100-110 microliters, 110-125 microliters, or 125-150 microliters may be used. Such larger volumes may permit administration of a higher dose as compared with a 100 microliter injection volume, without an increase in time required to deliver the dose and/or without requiring use of a lower gauge (wider diameter) needle. Such increase in total dose may be proportional to the increased volume. For example, an increase in dose volume of 50% would allow an increase of 50% in amount of compstatin analog administered without an increase in time required to deliver the dose and/or without requiring use of a lower gauge needle.

In certain embodiments a composition, e.g., a composition that may be used for IVT administration, e.g., for treatment of AMD, comprises about 150 mg/ml compstatin analog (e.g., comprising a PEG of about 40 kD), and one or more excipients selected from the group consisting of NaCl, trehalose, and sorbitol. In certain embodiments any of the compositions comprising about 150 mg/ml compstatin analog has a concentration between 140 mg/ml and 150 mg/ml, e.g., between 145 mg/ml and 155 mg/ml, e.g., between 148 mg/ml and 152 mg/ml, e.g., 150 mg/ml compstatin analog. In certain embodiments any of the compositions comprises about 20 mM sodium acetate. In certain embodiments any of the compositions has a pH between 4.8 and 5.2, e.g., 5.0.

In some embodiments the composition comprises about 150 mg/ml compstatin analog and between 0.45% and 0.60% w/v NaCl, e.g., between 0.45% and 0.50%, between 0.50% and 0.55%, or between 0.55% and 0.60% NaCl. In certain embodiments the composition comprises between 0.51% NaCl and 0.54% NaCl. In certain embodiments the composition comprises between 0.52% NaCl and 0.53% NaCl. In particular embodiments the composition comprises 0.500% NaCl. In particular embodiments the composition comprises 0.505% NaCl. 0.510% NaCl. In particular embodiments the composition comprises 0.515% NaCl. In particular embodiments the composition comprises 0.520% NaCl. In particular embodiments the composition comprises 0.525% NaCl. In particular embodiments the composition comprises s 0.530% NaCl. In particular embodiments the composition comprises 0.535% NaCl. In particular embodiments the composition contains 0.540% NaCl. In particular embodiments the composition comprises 0.545% NaCl. In particular embodiments the composition comprises 0.550% NaCl.

In certain embodiments the composition comprises about 150 mg/ml compstatin analog and between 2.5% and 4.5% w/v sorbitol w/v, e.g., between 2.5% and 3.0%, between 3.0% and 3.5%, between 3.5% and 4.0%, or between 4.0% and 4.5% sorbitol. In particular embodiments the composition comprises 3.0% sorbitol. In particular embodiments the composition comprises 3.1% sorbitol. In particular embodiments the composition comprises 3.2% sorbitol. In particular embodiments the composition comprises 3.3% sorbitol. In particular embodiments the composition comprises 3.4% sorbitol. In particular embodiments the composition comprises 3.5% sorbitol.

In certain embodiments the composition comprises about 150 mg/ml compstatin analog and between 6.0% and 8.0% w/v trehalose, e.g., between 6.0% and 6.5%, between 6.5% and 7.0%, between 7.0% and 7.5%, or between 7.5% and 8.0% trehalose. In particular embodiments the composition comprises 6.5% trehalose. In particular embodiments the composition comprises 6.6% trehalose. In particular embodiments the composition comprises 6.7% trehalose. In particular embodiments the composition comprises 6.8% trehalose. In particular embodiments the composition comprises 6.9% trehalose. In particular embodiments the composition comprises 7.0% trehalose.

In some embodiments, the osmolality of a solution comprising a compstatin analog may be measured at a dilution such that the concentration of compstatin analog is about 75 mg/ml or less. A solution comprising a compstatin analog and, optionally, an osmolality modifier, may be diluted with water by an appropriate dilution factor such that the concentration of the compstatin analog is 75 mg/ml or less, e.g., between 25 mg/ml and 40 mg/ml or between 40 mg/ml and 75 mg/ml. The osmolality of the resulting solution is measured and then multiplied by the dilution factor to correct for the dilution. For example, a solution having a compstatin analog concentration of 100 mg/ml may be diluted by a factor of 2, i.e., to a concentration of 50 mg/ml. The osmolality of the resulting solution is measured and multiplied by 2 to yield the osmolality of the original 100 mg/ml solution. As another example, a solution having a compstatin analog concentration of 150 mg/ml may be diluted by a factor of 2, i.e., to a concentration of 75 mg/ml. The osmolality of the resulting solution is measured and multiplied by 2 to yield the osmolality of the original 100 mg/ml solution.

In some embodiments a dose of a composition comprising a compstatin analog is administered by ocular administration (e.g., IVT injection) once a month, every 6 weeks, or every 2 months (i.e., every other month). In some embodiments a dose of a composition comprising a compstatin analog is administered by IVT injection every 3 months, every 4 months, every 5 months, or every 6 months, or less frequently, e.g., every 9 months, every year). Thus in some embodiments a patient may receive between 1 and 6 injections per year, typically at approximately equal intervals. In some embodiments a patient is initially treated with monthly injections (e.g., for the first 3-6 months or the first 6-12 months), followed by less frequent administration (e.g., every 2, 3, 4, 5, or 6 months, or less frequently, e.g., every 9 months, every year).

In some embodiments, treatment with a compstatin analog by IVT injection according to any of the afore-mentioned dosing regimens may be continued indefinitely. In some embodiments, a patient may be treated with a short course of treatment (e.g., 1, 2, 3, 4, 5, or 6 IVT injections). In some embodiments, a short course of treatment may be sufficient to halt or substantially halt progression of a disorder (e.g., AMD, e.g., GA or early or intermediate AMD), such that further treatment is not needed. For example, a short course of treatment with a compstatin analog may be sufficient to disrupt a cycle that perpetuates an immune response against the retina or retinal pigment epithelium.

In some embodiments a compstatin analog is administered using a syringe with one or more design features that reduce friction and/or required injection force, such as a relatively short barrel and/or relatively large plunger size.

In experiments performed using compstatin analogs comprising PEGs of different molecular weights, it was determined that a solution of compstatin analog comprising a lower molecular weight PEG (e.g., 10 kD-30 kD) has reduced viscosity as compared to a compstatin analog comprising a 40 kD PEG at the same concentration in mg/ml. Lower viscosity can facilitate use of a higher gauge number needle (e.g., 29 gauge rather than 27 gauge). In some embodiments a compstatin analog comprising a polymer, e.g., a PEG having a molecular weight below 40 kD, may be administered using a needle with a smaller inner diameter and/or higher gauge number than a compstatin analog comprising a polymer, e.g., a PEG having a molecular weight of 40 kD or more. In some embodiments a compstatin analog comprising a polymer, e.g., a PEG having a molecular weight below 40 kD, may be administered at a concentration between 80 mg/ml and 150 mg/ml, e.g., about 100 mg/ml or about 125 mg/ml. In some embodiments a compstatin analog comprising a polymer, e.g., a PEG, having a molecular weight below 40 kD, may be administered at a concentration between 150 mg/ml and 250 mg/ml. In some embodiments a compstatin analog comprising a polymer, e.g., a PEG having a molecular weight below 40 kD (e.g., 10 kD-35 kD), may be administered at a higher concentration than 250 mg/ml, e.g., up to 300 mg/ml, 300 mg/ml-400 mg/ml, 400 mg/ml-500 mg/ml, or more.

The disclosure encompasses administration of a compstatin analog in combination with additional therapy. Such additional therapy may include administration of any agent(s) used in the art or potentially useful for treating a subject suffering from the disease. For example, in some embodiments a compstatin analog is administered in combination with a C5 inhibitor (e.g., eculizumab or any of the other C5 inhibitors mentioned herein or known in the art) to a patient, e.g., a patient with AMD. In some embodiments a compstatin analog is administered in combination with an anti-vascular endothelial growth factor (VEGF) agent to a subject with wet AMD. Anti-VEGF agents include antibodies that bind to VEGF such as ranibizumab (Lucentis), bevacizumab (Avastin), brolucizumab (Novartis), polypeptides comprising a soluble portion of VEGF receptor such as aflibercept (Eylea, also known as VEGF-Trap).

When two or more therapies (e.g., compounds or compositions) are used or administered “in combination” with each other, they may be given at the same time, within overlapping time periods, or sequentially (e.g., separated by up to 2 weeks in time, or more, e.g., separated by up to about 4, 6, 8, or 12 weeks in time), in various embodiments of the invention. They may be administered via the same route or different routes. In some embodiments, the compounds or compositions are administered within 48 hours of each other. In some embodiments, a compstatin analog can be given prior to or after administration of the additional compound(s), e.g., sufficiently close in time that the compstatin analog and additional compound(s) are present at useful levels within the body at least once. In some embodiments, the compounds or compositions are administered sufficiently close together in time such that no more than 90% of the earlier administered composition has been metabolized to inactive metabolites or eliminated, e.g., excreted, from the body, at the time the second compound or composition is administered.

In some embodiments, a composition that includes both a compstatin analog and additional compound(s) is administered.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXEMPLIFICATION

### Example 1: Preclinical Studies of Intravitreal Pegcetacoplan

Preclinical studies in monkeys were performed to assess the safety and pharmacology of pegcetacoplan when injected intravitreally. Intravitreally administered pegcetacoplan in cynomolgus monkeys is distributed into the blood stream then further distributed and/or slowly eliminated from the body. The results of toxicokinetic analyses of vitreal and serum concentrations of pegcetacoplan after repeated intravitreal injections over the course of nine months at doses up to 24.8 mg/eye in 5% dextrose in volumes of either 50 or 100 μL/eye indicated that there was little intraocular or serum accumulation of the drug over multiple injections. In addition, a full toxicological review, including ophthalmological assessment by indirect and slit lamp, spectral domain optical coherence tomography, electroretinography, and tonometry and histopathological examinations of both eyes and of approximately 50 additional tissues from each monkey revealed no evidence of pegcetacoplan-mediated changes at any of the doses tested.

Assessment of the pharmacokinetic profile of a single intravitreal dose pegcetacoplan (10 mg/eye in both eyes) in monkeys revealed a vitreal half-life of approximately 3.2 days. After intravitreal injection, serum concentrations of pegcetacoplan increased until post-dose Day 7 then decreased with an apparent half-life of 10.4 days.

### Example 2: Phase 1b Single Ascending Dose Clinical Trial of Pegcetacoplan in Subjects with AMD

A Phase 1 open label, single ascending dose clinical trial of pegcetacoplan in patients with wet AMD and receiving anti-VEGF therapy (specifically, Lucentis®, Eylea® or Avastin®), was initiated to assess safety, tolerability and PK of pegcetacoplan. In this trial, patients receive a single dose of pegcetacoplan by intravitreal injection followed by 113 days of monitoring. It was originally planned to enroll nine patients in the trial, in three cohorts of three patients each, at doses of 5 mg, 10 mg and 20 mg of pegcetacoplan in 5% dextrose in a volume of 100 microliters. After enrollment of all three cohorts was completed, the third cohort was expanded from three patients to a total of 12 patients. pegcetacoplan was well tolerated in the initial nine patients, and no serious adverse events were reported.

The present disclosure provides dosing regimens in accordance with which a compstatin analog (e.g., pegcetacoplan) is administered by intravitreal injection. In some embodiments, pegcetacoplan is administered as sole therapy; in some embodiments, it is administered in combination with another therapy (e.g., anti-VEGF therapy), so that the patient is simultaneously exposed to both.

The present Example specifically describes and supports dosing regimens under which pegcetacoplan is administered by intravitreal injection to subjects who are receiving VEGF therapy. In some embodiments, subjects treated with both anti-VEGF therapy and therapy with pegcetacoplan receive doses of an anti-VEGF agent at intervals longer that those utilized for otherwise comparable subjects not receiving therapy with pegcetacoplan. A variety of anti-VEGF agents have been developed (reviewed, for example, in Lanzetta Br J Opthamol 97:1497, 2013). For example, reported dosing regimens for certain anti-VEGF agents include intravitreal injections of ranibizumab 0.5 mg or bevacizumab 1.25 mg administered every 4 weeks (q4) or PRN; in some embodiments, such regimens serve as appropriate reference regimens with respect to which anti-VEGF combination therapy regimens as described herein are assessed.

In light of the disclosure provided herein, including in this Example, those skilled in the art would appreciate that provided are certain combination therapy regimens, for example, in accordance with which each of pegcetacoplan and an anti-VEGF agent is administered intravitreally; in some embodiments, pegcetacoplan and anti-VEGF may be administered together in a single injection for certain (though not necessarily all) doses. In some embodiments, fewer doses of the anti-VEGF doses are administered in a selected period of time than are administered absent administration of pegcetacoplan.

### Example 3: Phase 2 Single Ascending Dose Clinical Trial of Pegcetacoplan in Subjects with Geographic Atrophy

A randomized, single-masked, sham-controlled clinical trial of pegcetacoplan in patients with GA was conducted. 246 patients aged ≥50 years were enrolled in the trial.

Patients in the trial had a diagnosis of GA of the macula secondary to age-related macular degeneration, confirmed within 14 days prior to randomization by the central reading center using Fundus Autofluorescence (FAF) images, as well as the following criteria: Total GA area must be ≥2.5 mm² and ≤17.5 mm² (1 and 7 disc areas [DA] respectively), determined by screening images of FAF.

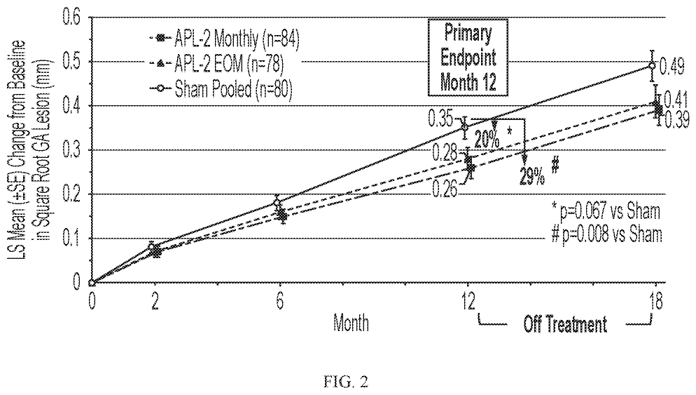

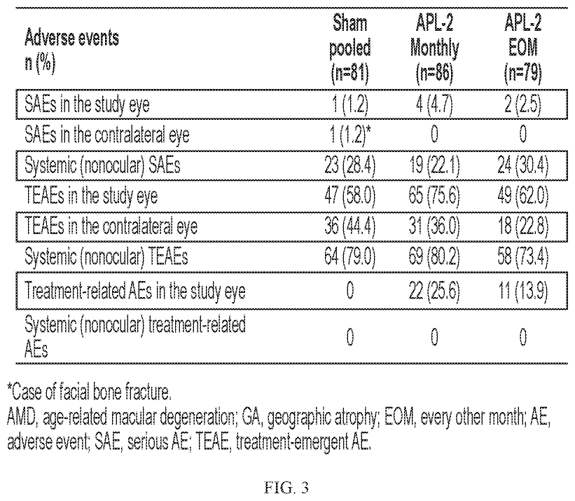

Patients were randomized in a 2:2:1:1 manner to receive 15 mg pegcetacoplan (APL-2) monthly, 15 mg pegcetacoplan every other month, sham injection monthly or sham injection every other month. Patients in the pegcetacoplan arms received a dose of 15 mg of pegcetacoplan, injected into the vitreous humor in a 0.1 cc volume, monthly or every other month for 12 months followed by six months of monitoring after the end of treatment. In the sham-injection cohorts, patients received a simulated injection. The safety, tolerability, PK, and evidence of activity of multiple intravitreal injections of pegcetacoplan in patients with GA in at least one eye were assessed. The primary efficacy endpoint was change in GA lesion size from baseline to month 12. As shown in FIG. 2, pegcetacoplan reduced rate of lesion growth in GA patients. The adverse event profile is depicted in FIG. 3. (See also Liao et al., Ophthalmology 127:186-195 (2020).)

Post hoc analysis was also conducted to assess disease progression outside of GA lesions. Participants in the study (n=246) were categorized into PM (n=86), EOM (n=79), and sham (n=81) categories. Eyes with missed injections and those that developed exudative AMD (whereupon pegcetacoplan treatment was discontinued) were excluded. Subjects from the pegcetacoplan monthly (PM; n=41), pegcetacoplan every other month (PEOM; n=56) and sham (S; n=70) arms, who completed Month 12 study visit and did not develop exudative AMD, were included (a total of 167 participants met the criteria for the post hoc analysis). Masked readers evaluated OCT scans outside a 500-micron perimeter from the GA border adhering to classification described in Sadda et al., Ophthalmology 125:537-548 (2018)). Specifically, to stay clear of the already manifested RPE or photoreceptor degeneration, areas outside a 500-μm perimeter from the GA border were analyzed, and up to five lesions were investigated within any given study eye. iRORA lesions identified at baseline were observed for progression to cRORA at subsequent timepoints. SD-OCT images obtained at baseline, Month 6, and Month 12 (using the Zeiss Cirrus or 156 Heidelberg Spectralis) using 7-line raster scans (30×5, 240 μm spacing, 7 sections, ART set to 25, 1536 A-scans) and volume scans (20×20; 49 sections, 120 μm spacing, ART set to 16, 512 A-scans) were graded following the CAM criteria independently by two masked readers at the Doheny Image Reading and Research Lab (DIRRL). A senior reader (and member of the CAM group) reviewed all questionable cases flagged by the readers and also adjudicated any discrepancies between graders in order to yield a single consensus result for each case.

Main outcome measures included assessments at baseline, Month 6 and Month 12 of: a) progression from large drusen (≥40 microns height) to incomplete/complete RPE and outer retina atrophy (iRORA and/or cRORA) and b) progression from iRORA to cRORA. Specifically, the endpoints for each treatment group were: proportion of study eyes with at least one iRORA at baseline that progressed to cRORA at 6 and 12 months, and proportion of individual baseline iRORA lesions that progressed to cRORA at 6 and 12 months.

Baseline characteristics were summarized using descriptive statistics. Continuous variables, such as GA lesion size, were summarized using the number of observations, mean, standard deviation, median, minimum, and maximum. Categorical variables, such as the presence of iRORA lesion(s) at baseline, were summarized using counts and percentages. The progression of iRORA to cRORA was summarized at Months 6 and 12 at both the study eye and lesion level. For the study-eye-level summaries, the subset of eyes with at least one iRORA lesion at baseline was included. If at least one iRORA lesion was present at baseline progressed to cRORA during follow-up, the eye was counted as having progressed. The proportion of study eyes with progression was compared between each pegcetacoplan group and the sham control group using a Pearson chi-square test. P values <0.05 were considered statistically significant. Risk ratios and their corresponding 95% confidence intervals were calculated as the proportion that progressed from iRORA to cRORA in the pegcetacoplan group divided by the proportion in the sham group. For the lesion-level summaries of progression, each iRORA lesion present at baseline was assessed separately for progression to a cRORA lesion; the proportion of lesions that progressed was compared between each pegcetacoplan group and the sham control group using descriptive statistics (counts and percentages).

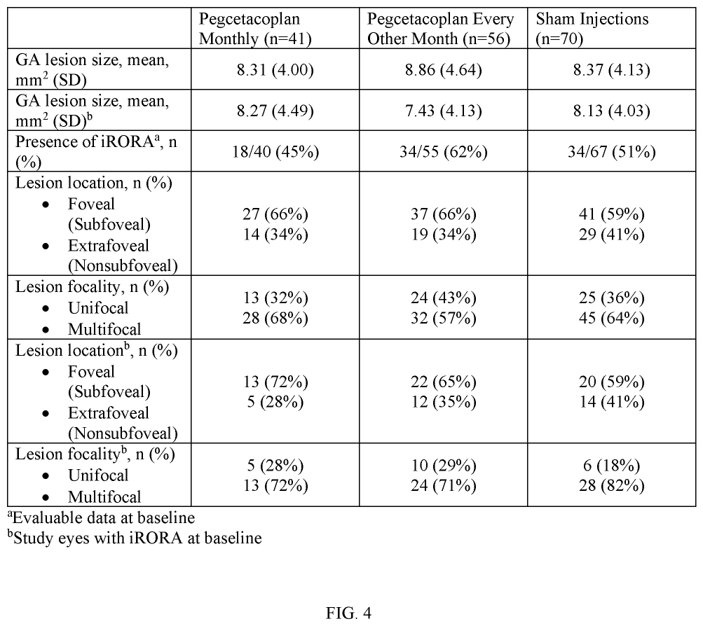

Large drusen were present at baseline in 81% (33/41) and 74% (49/66) of subjects in the PM and S groups, respectively. As shown in FIG. 4, iRORA were present at baseline in 45% (18/40) of subjects in the PM group, 62% (34/55) of subjects in the PEOM group, and 51% (34/67) of subjects in the S group. There were no major differences between groups for GA lesion size, location, and focality (see FIG. 4).

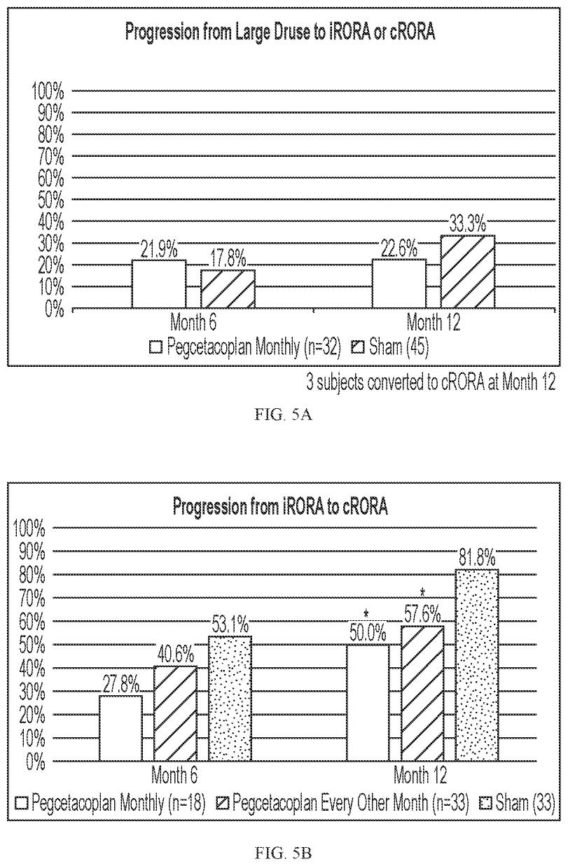

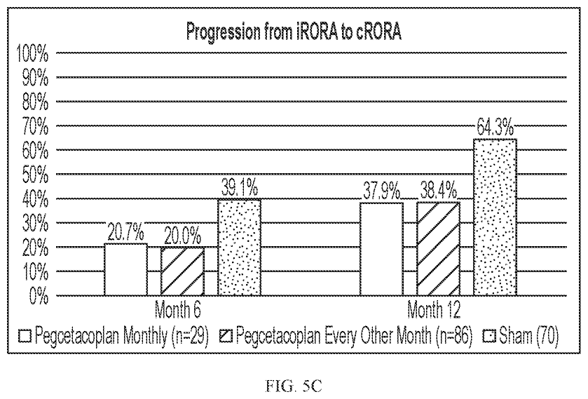

As shown in FIG. 5A, progression from large drusen to iRORA/cRORA at Month 12 was 22.6% (7/31) and 33.3% (15/45) in the PM and S groups, respectively (p=0.31). There were relatively fewer progression events at Month 12 in the pegcetacoplan group, and the trend suggested continued progression after Month 6 in the sham group but not in the pegcetacoplan group. As shown in FIG. 5B, progression from iRORA to cRORA was observed at month 6 in 27.8% (5/18), 40.6% (13/32), and 53.1% (17/32) in the PM, PEOM, and S groups, respectively (p=0.08 for PM and p=0.32 for EOM), and was observed at month 12 in 50.0% (9/18), 57.6% (19/33) and 81.8% (27/33) in the PM, PEOM, and S groups, respectively (p=0.02 for PM; p=0.03 for PEOM). Treatment with monthly pegcetacoplan resulted in a 39% reduction in the risk of progression to cRORA at 12 months compared with sham, and a 30% reduction for treatment with pegcetacoplan every other month. FIG. 5C shows progression from iRORA to cRORA using lesion level data. Similar to the findings at the study-eye level, at the lesion level, rates of progression to cRORA at month 6 were 20.7% (6/29), 20.0% (16/80), and 39.1% (27/69) in the PM, PEOM, and sham groups, respectively, and at month 12, rates of lesion progression up to a maximum of five lesions were 37.9% (11/29), 38.4% (33/86), and 64.3% (45/70) in the PM, EOM, and sham groups, respectively. Treatment with monthly pegcetacoplan resulted in a 41% reduction in the risk of progression to cRORA at 12 months compared with sham, and a 40% reduction for treatment with pegcetacoplan every other month. As indicated by FIGS. 5B and 5C, there was a lower rate of progression from iRORA to cRORA in pegcetacoplan-treated subjects compared to sham group.

These findings demonstrate that subjects receiving intravitreal injections of pegcetacoplan monthly or every other month had a lower rate of progression from nascent GA to GA when compared to sham controls.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. It will be appreciated that the invention is in no way dependent upon particular results achieved in any specific example or with any specific embodiment. Articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. For example, and without limitation, it is understood that where claims or description indicate that a residue at a particular position may be selected from a particular group of amino acids or amino acid analogs, the invention includes individual embodiments in which the residue at that position is any of the listed amino acids or amino acid analogs. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims or from the description above is introduced into another claim. For example, any claim that is dependent on another claim can be modified to include one or more elements, limitations, clauses, or descriptive terms, found in any other claim that is dependent on the same base claim. Furthermore, where the claims recite a composition, it is to be understood that methods of administering the composition according to any of the methods disclosed herein, and methods of using the composition for any of the purposes disclosed herein are included within the scope of the invention, and methods of making the composition according to any of the methods of making disclosed herein are included within the scope of the invention, unless otherwise indicated or unless it would be evident to one of ordinary skill in the art that a contradiction or inconsistency would arise. Methods of treating a subject can include a step of providing a subject in need of such treatment (e.g., a subject who has had, or is at increased risk of having, a disease), a step of diagnosing a subject as having a disease and/or a step of selecting a subject for treatment with a compstatin analog. Where elements are presented as lists, it is to be understood that each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc. Discussion of various diseases, disorders, and conditions under various headings herein is for convenience and is not intended to limit the invention.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Any particular embodiment, aspect, element, feature, etc., of the present invention may be explicitly excluded from the claims even if such exclusion is not set forth explicitly herein. For example, any compstatin analog, functional group, linking portion, clearance-reducing moiety, disease, or indication can be explicitly excluded.

We claim:

1. A method of treating a subject, comprising:
administering, to at least one eye of a subject determined to have one or more drusen, an effective amount of a C3 inhibitor,
wherein administering the C3 inhibitor reduces risk of the subject developing incomplete retinal pigment epithelium and outer retinal atrophy (iRORA) and/or complete retinal pigment epithelium and outer retinal atrophy (cRORA).

2. The method of claim 1, further comprising detecting one or more drusen in at least one eye of the subject.

3. The method of claim 1 or 2, wherein the one or more drusen are greater than 60 (e.g., greater than 70 or 80) microns in size.

4. The method of any one of claims 1-3, further comprising assessing the subject for one or more sign or symptom of iRORA or cRORA.

5. The method of claim 4, wherein the assessing step comprises optical coherence tomography (OCT) (e.g., swept-source OCT).

6. The method of any one of claims 1-5, wherein iRORA is determined by:
(i) detecting a region of signal hypertransmission into the choroid of less than about 250 μm in diameter;
(ii) detecting a corresponding zone of attenuation or disruption of the retinal pigment epithelium (RPE) of less than about 250 μm in diameter (e.g., with or without persistence of basal laminar deposits); and
(iii) detecting evidence of overlying photoreceptor degeneration (e.g., subsidence of the inner nuclear layer (INL) and outer plexiform layer (OPL), presence of a hyporeflective wedge in the Henle fiber layer (HFL), thinning of the outer nuclear layer (ONL), disruption of the external limiting membrane (ELM), and/or disintegrity of the ellipsoid zone (EZ)).

7. The method of any one of claims 1-6, wherein cRORA is determined by:
(i) detecting a region of hypertransmission of at least 250 μm in diameter;
(ii) detecting a zone of attenuation or disruption of the RPE of at least 250 μm in diameter; and
(iii) detecting evidence of overlying photoreceptor degeneration (e.g., subsidence of the INL and OPL, presence of a hyporeflective wedge in the HFL, thinning of the ONL, disruption of the ELM, and/or disintegrity of the EZ).

8. The method of any one of claims 1-7, wherein the C3 inhibitor is administered (e.g., monthly or every other month) for at least 6 months, 12 months, or more.

9. The method of any one of claims 1-8, wherein after administration of the C3 inhibitor for about 6 months, about 12 months, or more, the subject does not develop iRORA and/or cRORA.

10. The method of any one of claims 1-9, wherein the subject has or is suffering from early age-related macular degeneration (AMD) or intermediate AMD.

11. The method of any one of claims 1-10, wherein the C3 inhibitor is administered to the eye of the subject by intravitreal injection.

12. A method of treating a subject, comprising:
administering, to at least one eye of a subject determined to have incomplete retinal pigment epithelium and outer retinal atrophy (iRORA), an effective amount of a C3 inhibitor,
wherein administering the C3 inhibitor reduces risk of the subject developing complete retinal pigment epithelium and outer retinal atrophy (cRORA).

13. The method of claim 12, further comprising detecting incomplete retinal pigment epithelium and outer retinal atrophy (iRORA) in at least one eye of the subject.

14. The method of claim 12 or 13, further comprising assessing the subject for one or more sign or symptom of iRORA or cRORA.

15. The method of claim 14, wherein the assessing step comprises optical coherence tomography (OCT) (e.g., swept-source OCT).

16. The method of any one of claims 12-15, wherein iRORA is determined by:
(i) detecting a region of signal hypertransmission into the choroid of less than about 250 μm in diameter;
(ii) detecting a corresponding zone of attenuation or disruption of the retinal pigment epithelium (RPE) of less than about 250 μm in diameter (e.g., with or without persistence of basal laminar deposits); and
(iii) detecting evidence of overlying photoreceptor degeneration (e.g., subsidence of the inner nuclear layer (INL) and outer plexiform layer (OPL), presence of a hyporeflective wedge in the Henle fiber layer (HFL), thinning of the outer nuclear layer (ONL), disruption of the external limiting membrane (ELM), and/or disintegrity of the ellipsoid zone (EZ)).

17. The method of any one of claims 12-16, wherein cRORA is determined by:
(i) detecting a region of hypertransmission of at least 250 μm in diameter;
(ii) detecting a zone of attenuation or disruption of the RPE of at least 250 μm in diameter; and
(iii) detecting evidence of overlying photoreceptor degeneration (e.g., subsidence of the INL and OPL, presence of a hyporeflective wedge in the HFL, thinning of the ONL, disruption of the ELM, and/or disintegrity of the EZ).

18. The method of any one of claims 12-17, wherein the C3 inhibitor is administered (e.g., monthly or every other month) for at least 6 months, 12 months, or more.

19. The method of any one of claims 12-18, wherein after administration of the C3 inhibitor for about 6 months, about 12 months, or more, the subject does not develop cRORA.

20. The method of any one of claims 12-19, wherein the subject has or is suffering from early age-related macular degeneration (AMD) or intermediate AMD.

21. The method of any one of claims 12-20, wherein the C3 inhibitor is administered to the eye of the subject by intravitreal injection.

22. A method of slowing or preventing development of iRORA and/or cRORA in a subject, the method comprising:
administering, to at least one eye of a subject determined to have one or more drusen, an effective amount of a C3 inhibitor (e.g., monthly or every other month) for at least 6 months, 12 months, or more,
wherein after administration of the C3 inhibitor for at least 6 months, 12 months, or more, the subject does not develop iRORA and/or cRORA.

23. The method of claim 22, further comprising detecting one or more drusen in at least one eye of the subject.

24. The method of claim 22 or 23, wherein the one or more drusen are greater than 60 (e.g., greater than 70 or 80) microns in size.

25. The method of any one of claims 22-24, further comprising assessing the subject for one or more sign or symptom of iRORA or cRORA.

26. The method of claim 25, wherein the assessing step comprises optical coherence tomography (OCT) (e.g., swept-source OCT).

27. The method of any one of claims 22-26, wherein iRORA is determined by:
(i) detecting a region of signal hypertransmission into the choroid of less than about 250 μm in diameter;
(ii) detecting a corresponding zone of attenuation or disruption of the retinal pigment epithelium (RPE) of less than about 250 μm in diameter (e.g., with or without persistence of basal laminar deposits); and
(iii) detecting evidence of overlying photoreceptor degeneration (e.g., subsidence of the inner nuclear layer (INL) and outer plexiform layer (OPL), presence of a hyporeflective wedge in the Henle fiber layer (HFL), thinning of the outer nuclear layer (ONL), disruption of the external limiting membrane (ELM), and/or disintegrity of the ellipsoid zone (EZ)).

28. The method of any one of claims 22-27, wherein cRORA is determined by:
(i) detecting a region of hypertransmission of at least 250 μm in diameter;
(ii) detecting a zone of attenuation or disruption of the RPE of at least 250 μm in diameter; and
(iii) detecting evidence of overlying photoreceptor degeneration (e.g., subsidence of the INL and OPL, presence of a hyporeflective wedge in the HFL, thinning of the ONL, disruption of the ELM, and/or disintegrity of the EZ).

29. The method of any one of claims 22-28, wherein the subject has or is suffering from early age-related macular degeneration (AMD) or intermediate AMD.

30. The method of any one of claims 22-29, wherein the C3 inhibitor is administered to the eye of the subject by intravitreal injection.

31. A method of slowing or preventing development of cRORA in a subject, the method comprising:
administering, to at least one eye of a subject determined to have iRORA, an effective amount of a C3 inhibitor (e.g., monthly or every other month) for at least 6 months, 12 months, or more,
wherein after administration of the C3 inhibitor for at least 6 months, 12 months, or more, the subject does not develop cRORA.

32. The method of claim 31, further comprising detecting iRORA in at least one eye of the subject.

33. The method of claim 31 or 32, further comprising assessing the subject for one or more sign or symptom of iRORA or cRORA.

34. The method of claim 33, wherein the assessing step comprises optical coherence tomography (OCT) (e.g., swept-source OCT).

35. The method of any one of claims 31-34, wherein iRORA is determined by:
(i) detecting a region of signal hypertransmission into the choroid of less than about 250 μm in diameter;
(ii) detecting a corresponding zone of attenuation or disruption of the retinal pigment epithelium (RPE) of less than about 250 μm in diameter (e.g., with or without persistence of basal laminar deposits); and
(iii) detecting evidence of overlying photoreceptor degeneration (e.g., subsidence of the inner nuclear layer (INL) and outer plexiform layer (OPL), presence of a hyporeflective wedge in the Henle fiber layer (HFL), thinning of the outer nuclear layer (ONL), disruption of the external limiting membrane (ELM), and/or disintegrity of the ellipsoid zone (EZ)).

36. The method of any one of claims 31-35, wherein cRORA is determined by:
(i) detecting a region of hypertransmission of at least 250 μm in diameter;
(ii) detecting a zone of attenuation or disruption of the RPE of at least 250 μm in diameter; and
(iii) detecting evidence of overlying photoreceptor degeneration (e.g., subsidence of the INL and OPL, presence of a hyporeflective wedge in the HFL, thinning of the ONL, disruption of the ELM, and/or disintegrity of the EZ).

37. The method of any one of claims 31-36, wherein the subject has or is suffering from early age-related macular degeneration (AMD) or intermediate AMD.

38. The method of any one of claims 31-37, wherein the C3 inhibitor is administered to the eye of the subject by intravitreal injection.

39. A method of slowing or preventing development of iRORA and/or cRORA in a subject, the method comprising:
providing, to a subject determined to have one or more drusen in at least one eye, a treatment regimen, wherein the treatment regimen comprises administering to the at least one eye of the subject an effective amount of a C3 inhibitor (e.g., monthly or every other month) for at least 6 months, 12 months, or more,
wherein the subject does not develop iRORA and/or cRORA for at least 6 months, 12 months, 18 months, 24 months, 30 months, 36 months, or more, after starting the treatment regimen.

40. The method of claim 39, further comprising detecting one or more drusen in at least one eye of the subject.

41. The method of claim 39 or 40, wherein the one or more drusen are greater than 60 (e.g., greater than 70 or 80) microns in size.

42. The method of any one of claims 39-41, further comprising assessing the subject for one or more sign or symptom of iRORA or cRORA.

43. The method of claim 42, wherein the assessing step comprises optical coherence tomography (OCT) (e.g., swept-source OCT).

44. The method of any one of claims 39-43, wherein iRORA is determined by:
(i) detecting a region of signal hypertransmission into the choroid of less than about 250 μm in diameter;
(ii) detecting a corresponding zone of attenuation or disruption of the retinal pigment epithelium (RPE) of less than about 250 μm in diameter (e.g., with or without persistence of basal laminar deposits); and
(iii) detecting evidence of overlying photoreceptor degeneration (e.g., subsidence of the inner nuclear layer (INL) and outer plexiform layer (OPL), presence of a hyporeflective wedge in the Henle fiber layer (HFL), thinning of the outer nuclear layer (ONL), disruption of the external limiting membrane (ELM), and/or disintegrity of the ellipsoid zone (EZ)).

45. The method of any one of claims 39-44, wherein cRORA is determined by:
(i) detecting a region of hypertransmission of at least 250 μm in diameter;
(ii) detecting a zone of attenuation or disruption of the RPE of at least 250 μm in diameter; and
(iii) detecting evidence of overlying photoreceptor degeneration (e.g., subsidence of the INL and OPL, presence of a hyporeflective wedge in the HFL, thinning of the ONL, disruption of the ELM, and/or disintegrity of the EZ).

46. The method of any one of claims 39-45, wherein the subject has or is suffering from early age-related macular degeneration (AMD) or intermediate AMD.

47. The method of any one of claims 39-46, wherein the C3 inhibitor is administered to the eye of the subject by intravitreal injection.

48. A method of slowing or preventing development of cRORA in a subject, the method comprising:
providing, to a subject determined to have iRORA in at least one eye, a treatment regimen, wherein the treatment regimen comprises administering to the at least one eye of the subject an effective amount of a C3 inhibitor (e.g., monthly or every other month) for at least 6 months, 12 months, or more,
wherein the subject does not develop cRORA for at least 6 months, 12 months, 18 months, 24 months, 30 months, 36 months, or more, after starting the treatment regimen.

49. The method of claim 48, further comprising detecting iRORA in at least one eye of the subject.

50. The method of claim 48 or 49, further comprising assessing the subject for one or more sign or symptom of iRORA or cRORA.

51. The method of claim 50, wherein the assessing step comprises optical coherence tomography (OCT) (e.g., swept-source OCT).

52. The method of any one of claims 48-51, wherein iRORA is determined by:
(i) detecting a region of signal hypertransmission into the choroid of less than about 250 μm in diameter;
(ii) detecting a corresponding zone of attenuation or disruption of the retinal pigment epithelium (RPE) of less than about 250 μm in diameter (e.g., with or without persistence of basal laminar deposits); and
(iii) detecting evidence of overlying photoreceptor degeneration (e.g., subsidence of the inner nuclear layer (INL) and outer plexiform layer (OPL), presence of a hyporeflective wedge in the Henle fiber layer (HFL), thinning of the outer nuclear layer (ONL), disruption of the external limiting membrane (ELM), and/or disintegrity of the ellipsoid zone (EZ)).

53. The method of any one of claims 48-52, wherein cRORA is determined by:
(i) detecting a region of hypertransmission of at least 250 μm in diameter;
(ii) detecting a zone of attenuation or disruption of the RPE of at least 250 μm in diameter; and
(iii) detecting evidence of overlying photoreceptor degeneration (e.g., subsidence of the INL and OPL, presence of a hyporeflective wedge in the HFL, thinning of the ONL, disruption of the ELM, and/or disintegrity of the EZ).

54. The method of any one of claims 48-53, wherein the subject has or is suffering from early age-related macular degeneration (AMD) or intermediate AMD.

55. The method of any one of claims 48-54, wherein the C3 inhibitor is administered to the eye of the subject by intravitreal injection.

56. A method of slowing or preventing development of iRORA and/or cRORA in a subject for at least 6 months, 12 months, 18 months, 24 months, 30 months, 36 months, or more, after providing a treatment regimen to the subject, the method comprising:
administering, to a subject determined to have one or more drusen in at least one eye, the treatment regimen, wherein the treatment regimen comprises administering to the at least one eye of the subject an effective amount of a C3 inhibitor (e.g., monthly or every other month) for at least 6 months, 12 months, or more,
thereby slowing or preventing development of iRORA and/or cRORA for at least 6 months, 12 months, 18 months, 24 months, 30 months, 36 months, or more, after starting the treatment regimen.

57. The method of claim 56, further comprising detecting one or more drusen in at least one eye of the subject.

58. A method of slowing or preventing development of cRORA in a subject for at least 6 months, 12 months, 18 months, 24 months, 30 months, 36 months, or more, after providing a treatment regimen to the subject, the method comprising:
administering, to a subject determined to have iRORA in at least one eye, the treatment regimen, wherein the treatment regimen comprises administering to the at least one eye of the subject an effective amount of a C3 inhibitor (e.g., monthly or every other month) for at least 6 months, 12 months, or more,
thereby slowing or preventing development of cRORA for at least 6 months, 12 months, 18 months, 24 months, 30 months, 36 months, or more, after starting the treatment regimen.

59. The method of claim 58, further comprising detecting iRORA in at least one eye of the subject.

60. The method of any of the preceding claims, wherein the C3 inhibitor comprises a compstatin analog comprising at least one compstatin analog moiety.

61. The method of claim 60, wherein the compstatin analog comprises a clearance reducing moiety (CRM) and at least one compstatin analog moiety.

62. The method of claim 61, wherein the compstatin analog comprises a CRM having at least two compstatin analog moieties attached thereto.

63. The method of claim 61 or 62, wherein the CRM comprises a PEG.

64. The method of any one of claims 61-63, wherein the CRM has an average molecular weight of between about 10 kD and about 50 kD.

65. The method of any one of claims 61-64, where the CRM has an average molecular weight between about 35 kD and about 45 kD, e.g., about 40 kD.

66. The method of any one of claims 60-65, wherein the compstatin analog comprises a linear polymer having a compstatin analog moiety attached to each end.

67. The method of any one of claims 60-66, wherein each compstatin analog moiety comprises a cyclic peptide that comprises the amino acid sequence of one of SEQ ID NOs: 3-36, optionally selected from SEQ ID Nos: 9-36.

68. The method of any one of claims 60-67, wherein each compstatin analog moiety comprises a cyclic peptide that comprises an amino acid sequence as set forth in any of SEQ ID NOs: 28, 32, or 34.

69. The method of any one of claims 60-68, wherein each compstatin analog moiety comprises a cyclic peptide comprising a cyclic portion 11 amino acids in length, wherein the sequence of the peptide is at least 50% identical to the sequence of compstatin (SEQ ID NO: 8) or to the sequence of a compstatin analog that has higher activity than compstatin.

70. The method of any one of claims 60-69, wherein each compstatin analog moiety comprises a cyclic peptide comprising a cyclic portion 11 amino acids in length, wherein the sequence of the peptide is at least 50% identical to the sequence of compstatin (SEQ ID NO: 8) or to the sequence of a compstatin analog that has higher activity than compstatin, wherein the peptide comprises at least one non-standard amino acid, optionally wherein at least one non-standard amino acid is a singly or multiply halogenated amino acid, N-alkyl amino acid, or aromatic amino acid.

71. The method of any one of claims 60-70, wherein each compstatin analog moiety comprises a peptide comprising a sequence that has 1, 2, 3, or 4 substitutions relative to the sequence of compstatin, wherein the peptide is cyclized via a bond between amino acids at positions that correspond to position 2 and position 12 of compstatin, wherein 1, 2, 3, or 4 amino acids in the sequence of compstatin is replaced by a non-standard amino acid, and wherein optionally at least one non-standard amino acid is a singly or multiply halogenated amino acid, N-alkyl amino acid, or aromatic amino acid.

72. The method of any one of claims 60-71, wherein the compstatin analog comprises one or more compstatin analog moiet(ies) that comprise a cyclic peptide having a 1-methylTrp at a position corresponding to position 4 of SEQ ID NO:8.

73. The method of any one of claims 60-72, wherein the compstatin analog comprises one or more compstatin analog moiet(ies) that comprise a cyclic peptide having an N-methylGly at a position corresponding to position 8 of SEQ ID NO:8.

74. The method of any one of claims 60-73, wherein the compstatin analog comprises one or more clearance-reducing moieties attached to one or more compstatin analog moieties, wherein: each compstatin analog moiety comprises a cyclic peptide having an amino acid sequence as set forth in any of SEQ ID NOs: 9-36, extended by one or more terminal amino acids at the N-terminus, C-terminus, or both, wherein one or more of the amino acids has a side chain comprising a primary or secondary amine and is separated from the cyclic peptide by a rigid or flexible spacer optionally comprising an oligo(ethylene glycol) moiety; and each clearance-reducing moiety optionally comprises a polyethylene glycol (PEG), wherein each clearance-reducing moiety is covalently attached via a linking moiety to one or more compstatin analog moieties, and wherein the linking moiety comprises an unsaturated alkyl moiety, a moiety comprising a nonaromatic cyclic ring system, an aromatic moiety, an ether moiety, an amide moiety, an ester moiety, a carbonyl moiety, an imine moiety, a thioether moiety, and/or an amino acid residue.

75. The method of any one of claims 60-74, wherein the compstatin analog comprises a clearance-reducing moiety attached to two compstatin analog moieties and wherein: (a) each compstatin analog moiety comprises a cyclic peptide extended by one or more amino acids at the N-terminus, C-terminus, or both, wherein the one or more amino acids is separated from the cyclic portion of the peptide by a rigid or flexible spacer, optionally wherein the spacer comprises an oligo(ethylene glycol) moiety; and (b) the clearance reducing moiety comprises a linear polymer, wherein each end of the linear polymer is linked to one of the compstatin analog moieties by way of a linker moiety comprising a carbonyl group.

76. The method of any one of claims 60-75, wherein the compstatin analog comprises a clearance-reducing moiety attached to two compstatin analog moieties and wherein: (a) each compstatin analog moiety comprises a cyclic peptide extended by one or more amino acids at the N-terminus, C-terminus, or both, wherein the one or more amino acids is separated from the cyclic portion of the peptide by a rigid or flexible spacer, optionally wherein the spacer comprises an oligo(ethylene glycol) moiety; and (b) the clearance reducing moiety comprises a linear polymer, wherein each end of the linear polymer is linked to one of the compstatin analog moieties by way of a carbamate.

77. The method of any of any one of claims 60-76, wherein each compstatin analog moiety comprises a cyclic peptide extended by an amino acid sequence that comprises at least one amino acid that has a side chain comprising a primary or secondary amine, optionally wherein the at least one amino acid that has a side chain comprising a primary or secondary amine is a lysine at the C-terminus of the cyclic peptide.

78. The method of any one of claims 60-77, wherein each compstatin analog moiety comprises a cyclic peptide extended by one or more amino acids at the N-terminus, C-terminus, or both, wherein the one or more amino acids is separated from the cyclic portion of the peptide by a rigid or flexible spacer that comprises an oligo(ethylene glycol) moiety, wherein the oligo(ethylene glycol) moiety is (—(O—CH₂—CH₂-)n, wherein n is between 1 and 10.

79. The method of any one of claims 60-78, wherein each compstatin analog moiety comprises a cyclic peptide extended by one or more amino acids at the N-terminus, C-terminus, or both, wherein the one or more amino acids is separated from the cyclic portion of the peptide by a rigid or flexible spacer that comprises —(CH₂)m- and —(O—CH₂—CH₂-)n joined covalently, wherein m is between 1 and 10 and n is between 1 and 10, optionally wherein m is 1 and n is 2.

80. The method of any one of claims 60-79, wherein each compstatin analog moiety comprises a cyclic peptide extended by one or more amino acids at the N-terminus, C-terminus, or both, wherein the one or more amino acids is separated from the cyclic portion of the peptide by a rigid or flexible spacer that comprises 8-amino-3,6-dioxaoctanoic acid (AEEAc) or 11-amino-3,6,9-trioxaundecanoic acid.

81. The method of claim 60, wherein the compstatin analog comprises a compound having the structure of FIG. 1.

82. The method of claim 81, wherein the compound comprises a PEG having an average molecular weight of about 40 kD.

83. The method of any one of claims 1-82, wherein the C3 inhibitor is administered as a composition comprising trehalose.

84. The method of claim 83, wherein the C3 inhibitor is administered at a dose of about 10 mg to about 20 mg, e.g., about 15 mg.
